(19) United States
(12) Patent Application Publication
(10) Pub. No.: US 2026/0209669 A1
(43) Pub. Date: Jul. 23, 2026

(54) NANOFIBROUS SCAFFOLD FOR CELL CULTURING

(71) Applicant: Cellevate AB, Lund (SE)

(72) Inventors: Albin Jakobsson, Lund (SE); Maximilian Ottosson, Lund (SE); Henrik Bratt, Lund (SE); Ulrik Flensborn, Lund (SE); Hira Kausar, Lund (SE)

(21) Appl. No.: 19/140,978

(22) PCT Filed: Dec. 21, 2023
(86) PCT No.: PCT/EP2023/087289

§ 371 Date: Jun. 18, 2025

(30) Foreign Application Priority Data
Dec. 21, 2022 (SE) 2251522-5

(51) Int. Cl. C12M 1/12; C12M 1/26; C12N 5/00; D01F 2/00

(52) U.S. Cl. CPC C12M 25/14; C12M 25/02; C12M 33/00; C12N 5/0068; D01F 2/00; C12N 2513/00; C12N 2533/78

(57) ABSTRACT

The present invention relates to a method for preparing a nanofibrous scaffold and use of the same for promoting cell culturing. In particular, the nanofibrous scaffold comprises functionalized cellulose nanofibers which significantly increases expansion capacity and protein production. The method comprises the step of dividing the initial cellulose nanofibers by dispersing.

### TECHNICAL FIELD OF THE INVENTION

The present invention relates to a method for preparing a nanofibrous scaffold and use of the same for promoting cell culturing. In particular, the nanofibrous scaffold comprises functionalized cellulose nanofibers which significantly increases expansion capacity and protein production.

### BACKGROUND OF THE INVENTION

Biological products (biologics), such as therapeutic proteins, vaccines, or cell and gene therapy products represent a new medical paradigm. Those therapies can provide remarkable outcomes for patients and have already revolutionized the treatment of many diseases in a variety of fields. The most complex biological products with specific glycosylation patterns may be produced by recombinant DNA technology in mammalian cell cultures and includes enzymes, synthetic hormones and monoclonal antibodies. However, manufacturing of biologics is challenging because production of end-products is reliant on sensitive live host cells which demands much more attention than e.g. chemical synthesis of small-molecule drugs.

The biologics manufacturing process can be separated into upstream processes and downstream processes. The upstream process is defined as the entire process from early cell isolation and cultivation to cell banking and culture expansion of the cells until final harvest—in other words the termination of the culture and collection of the live cell batch for purification.

While culturing of cells is therefore the crux of the upstream process, cell immunogenicity, adverse events, and efficacy can all be affected by even the slightest manufacturing process change. As such, it is very difficult to scale up from research to clinical quantities and many promising therapies can not reach the market. In addition, the most significant drawback is low product yield, followed by the costs related to the extensive cell-line development and limited cell viability for production. In all, the manufacturing process is extremely expensive and biologics are not accessible to all patients.

To assist the upstream process, microcarriers are routinely used to support proliferation of adherent cell populations in bioreactors. Microcarriers are support matrices that allow cells to be cultured in three dimensions instead of a traditional flat surface, leading to markedly increase in capacity to accommodate more cells in limited volumes. The most utilized commercially available microcarriers are solid spherical particles that retain the cells on their surface. This approach has accelerated production output as the cell growth is largely connected with available surface area for cell adhesion.

Suspension based bioreactors are preferred for many practical applications because they allow addition of more microcarriers and medium during the culturing process, which gives flexibility and control over the process. In the suspension based bioreactor, the microcarriers are freely floating in the bioreactor but provides the necessary support matrix for adherent to attach and proliferate. Important parameters for the microcarrier include the available surface area they provide and the dead volume they occupy.

Typically, a means of agitation is installed in the suspension based bioreactor to ensure that nutrients and gases, such as oxygen, are efficiently distributed in the bioreactor.

Agitation does however come with the drawback that high stirring speed may cause harmful collisions between the microcarriers or against the bioreactor, while low stirring speed may prevent the necessary distribution of nutrient and gases, both leading to sub-optimal cell growth. Accordingly, fluid dynamic forces in the closed system bioreactor plays an important role in not adversely affecting cell proliferation or risk damaging cells adhered to the microcarriers.

Thus, scale-up of cell growth for biologics production is still non-trivial as large quantities of high quality cells are required at a cost-effective mark if any commercialization is to be feasible. At present, there is a common understanding that not only the available surface area, but also the local spatial environment that the cells experience during culture is important for the magnitude and quality of the end cellular product. If the cells are not exposed to sufficient attachment sites and a microenvironment mimicking their natural surroundings, then cell signaling will be hampered and the cells will differentiate poorly.

Simply increasing the concentration of microcarriers in the bioreactor to increase available surface area and attachment points is not a viable solution since it would lead to more collisions between solid spherical microcarrier and incrementally increasing stress and damage to the cells. Moreover, high concentration of microcarriers have in some instances been shown to increase cell metabolite production, leading to faster consumption of culture medium and a deleterious growth environment and cytotoxicity. Accordingly, increasing the microcarrier does not necessarily translate into enhanced cell expansion rates.

With biomanufacturing of biologics evolving rapidly there is shaping a picture that scaling of production will be the bottleneck of the biologics' revolution in the coming years. However, the design of the existing microcarriers limits the available surface area and does not present the in-vivo like environment needed by many mammalian cells to proliferate efficiently. Effective microcarriers are at the forefront of solving this problem and assist in accelerating commercialization of biologics.

Thus, there is an unmet need for provision of new improved microcarriers that provides a high quality and scalable option for cell culturing.

Hence, it would be advantageous to provide a sustainable microcarrier material that offers an increased surface area to the cells in culture while at the same time replicates the extracellular environment found in the human body.

Specifically, it would be advantageous to provide a simple and scalable method for producing an improved microcarrier material that may be utilised all the way from stock culture to bioreactor production.

### SUMMARY OF THE INVENTION

The microcarriers presented herein are based on cellulose nanofibers that in processed form provide a matrix with an increased surface area for cell growth and low dead volume compared to competing solutions on the market. The cellulose nanofibers offer a scaffold matrix with physical properties that mimic the collagen and elastin fiber structures that make up the human extracellular matrix. Accordingly, cells grown on the microcarrier presented herein adopt physiological characteristics which resemble the native tissue from which they originate, leading to improved intercellular communications, which in turn results in a recovery or maintenance of in vivo functions.

Thus, an object of the present invention relates to the provision of a microcarrier with the capacity to increase growth of cells at minimal volume usage.

Another object of the present invention relates to provision of a simple method for producing an improved microcarrier that is scalable for industrial use.

Thus, an aspect of the present invention relates to a method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) functionalizing the processed cellulose nanofiber material by addition of a reagent comprising a functional moiety, and
(iv) drying the processed cellulose nanofiber material,
   thereby providing the nanofibrous cellulose scaffold.

Another aspect of the present invention relates to a nanofibrous cellulose scaffold obtainable from a method as described herein.

Yet another aspect of the present invention relates to a nanofibrous cellulose scaffold comprising a cellulose nanofiber material with a mean length of the cellulose nanofibers of less than about 250 μm, and wherein the cellulose nanofiber material is functionalized with a functional moiety.

A further aspect of the present invention relates to a microcarrier comprising a nanofibrous cellulose scaffold as described herein.

A still further aspect of the present invention relates to use of a nanofibrous cellulose scaffold as described herein as a microcarrier for cell culturing.

An even further aspect of the present invention relates to a cell culturing device comprising a container loaded with a nanofibrous cellulose scaffold or a microcarrier as described herein.

Another aspect of the present invention relates to a method for cell culturing comprising the steps of:
(i) providing a cell culturing device as described herein,
(ii) adding a composition comprising a cell population to the cell culturing device,
(iii) incubating the cell population to provide a proliferated cell population, and
(iv) optionally, extracting the proliferated cell population from the cell culturing device.

### BRIEF DESCRIPTION OF THE FIGURES

FIG. 1 shows (A) microscopy image of nanofibrous cellulose scaffold functionalized with quaternary ammonium (Cellevate QA). (B) Falcon tube comprising the nanofibrous cellulose scaffold in dry powder form. (C) Scanning electron microscopy (SEM) image of the nanofibrous cellulose scaffold demonstrating the spatial arrangement of individual nanofibers. (D) HEK293 cells cultured on the nanofibrous cellulose scaffold. (E) HEK 293 cells cultured on Cytodex-1 beads.

FIG. 2 shows cell growth of (A) HEK293 cells and (B) ARN8 cells on different microcarriers. The number of cells was quantified by lactate dehydrogenase activity. Cells were quantified on the day of seeding (day 0) and subsequent days to determine the growth capacity on the different microcarriers. The amount of microcarrier was adjusted to present the same available surface area to the cells irrespective of the type of microcarrier.

FIG. 3 shows luciferase production of (A) HEK293 cells and (B) ARN8 cells grown on different microcarriers. The amount of microcarrier was adjusted to present the same available surface area to the cells irrespective of the type of microcarrier.

FIG. 4 shows production of luciferase in HEK293 cells when taking into account the volume occupied by the different microcarriers (i.e. dead volume). (A) The microcarriers were decanted, with the grey line indicate the approximate volume of the microcarriers (from left to right; Cellevate CMC (“52”), Cellevate QA (“53”) and Cytodex-1). An equal volume of microcarrier is used. (B) Luciferase production after 24 hours (black bar) and 48 hours (grey bar) of cells grown on different microcarriers (equal dead volume).

FIG. 5 shows examples of fibers cut by laser. (A) The cellulose material cut by laser results in pieces of cellulose that trap air bubbles and float. (B) Electrospun cellulose material cut with a laser. The laser burns the cellulose sheets. (C) SEM images of cellulose nanofibers cut by laser. The cellulose nanofibers are melted and fused together.

FIG. 6 shows microscopy images of cellulose nanofibers divided by blending (A-E) or by dispersing (F-J) for different amounts of times.

FIG. 7 shows the difference between blending and dispersing the cellulose nanofibers. (A) Nanofiber length as a function of means of dividing and the duration of dividing (0.5-10 min). Nanofiber length is determined from SEM images using ImageJ software. (B) Representative SEM image of cellulose nanofibers after 10 minutes of blending. (C) Representative SEM image of cellulose nanofibers after 10 minutes of dispersing.

FIG. 8 shows determination of cellulose nanofiber diameters. (A-B) SEM micrographs of cellulose nanofibers after 1 hour dispersing at 18000 rpm. (C) Histogram depicting the cellulose nanofiber diameter distribution of five separate samples. Approximately 2500 individual cellulose nanofibers were measured using ImageJ software.

FIG. 9 shows scanning electron microscopy (SEM) images of electrospun nanofibers; (A) PCL, (B) PLA, (C) PLA/PCL, and (D) cellulose. All images are recorded at 600× magnification, scale bar is 50 μm.

FIG. 10 shows representative photographs (left) and SEM images (right) of different electrospun nanofibers subsequent to 5 minutes of mixing in a blender. (A-B) PCL, (C-D) PLA, (E-F) PLA/PCL. Scale bar of SEM images is 200 μm.

FIG. 11 shows representative photographs (left) and SEM images (right) of different electrospun nanofibers subsequent to 5 minutes of mixing in a disperser. (A-B) PCL, (C-D) PLA, (E-F) PLA/PCL. Scale bar of SEM images is 50 μm.

FIG. 12 shows scanning electron microscopy (SEM) images of cellulose nanofibers blended (left) or dispersed (right). The cellulose nanofibers were mixed for different periods of time; 1 min (A-B), 5 min (C-D), 15 min (E-F) or 60 min (G-H).

FIG. 13 shows histograms of size (length) distributions of cellulose nanofibers that have been divided by (A) blending or (B) dispersing. The histograms are for samples divided for 1, 5, 15, or 60 min (left to right). Nanofiber lengths are displayed as relative frequency of mean nanofiber lengths in bins of 200 μm.

FIG. 14 shows how cells behave on the nanofibrous cellulose scaffold. (A) Cell growth after 72 hours as function of degree of functionalization given as the titration value (mmol/CI). Time-course quantification of (B) concentration and (C) viability of HEK293 cells cultured on nanofibrous cellulose scaffolds prepared by dispersing the cellulose nanofibers for 1, 5, 15 or 60 minutes. Data are represented as means, with each data point representing a technical replicate (n=3).

### DETAILED DESCRIPTION OF THE INVENTION

### Definitions

Prior to outlining the present invention in more details, a set of terms and conventions is first defined:

### Nanofiber

In the present context, the term “nanofiber” refers to fibers with diameters in the range of 10-2000 nm. The fibers may be generated from different types of polymers, such as cellulose.

### Cellulose Nanofiber Material

In the present context, the term “cellulose nanofiber material” refers to an initial material prepared from cellulose. The cellulose nanofiber material may be prepared by any method suitable for preparing sheets of cellulose that can subsequently be processed as described herein to provide the nanofibrous cellulose scaffold. These methods include, but are not limited to, electrospinning, meltblowing, and drawing of cellulose nanofibers.

Preferably, the cellulose nanofiber material is prepared by electrospinning of cellulose nanofibers. Electrospinning may be performed from a solution of cellulose acetate.

### Container

In the present context, the term “container” refers to any delimited vessel suitable for culturing of cells. The container is preferably a conventional culturing vessel, including, but not limited to, a bioreactor, a cell culturing plate, a cell culture bottle, a spinner flask, a shaker flask, and a roller bottle, a petri dish, and a tube.

The nanofibrous cellulose scaffold as described herein is readily scalable and the container may therefore be of any volume suitable for culturing cells.

### Dispersing

In the present context, the term “dispersing” refers to the process of dividing the cellulose nanofiber material by means of a disperser. Dispersing is preferably performed in a liquid.

In the present context, a disperser is a high-speed mixing device that can comminute a solid, such as a cellulose sheet, into smaller fragments. The disperser comprises one or more heads which constitute the means of dividing the cellulose nanofiber material. The head may be in the form of a disc blade. The head of the disperser may comprise a blade with a propeller design. The disperser creates a turbulent flow and a vortex which ensures homogenous dividing of the cellulose nanofiber material into smaller fragments, i.e. cellulose nanofibers of reduced length compared to the initial cellulose nanofiber material.

Any type of disperser may be used, including, but not limited to, laboratory high-speed disperser, pilot-scale high-speed dispersers, and production-scale high-speed dispersers. The disperser can be chosen according to the batch size to be divided, and the diameter of the disperser blades are adjusted accordingly. The disperser blade may be raised and lowered during dispersing to eliminate stratification, such mechanism may be automatic.

For large batches the disperser may be a floor-mounted or tank-mounted model and/or be a multishaft model.

### Functional Moiety

In the present context, the term “functional moiety” refers to a chemical or biological group or molecule positioned on the nanofibrous cellulose scaffold, and which interacts with the cells associated with scaffold. A functional moiety may interact with the cells via interactions including, but not limited to, electrostatic interactions, affinity, and hydrophobicity/hydrophilicity. The functional moiety may promote attachment/adhesion of cells to the nanofibrous cellulose scaffold, induce cell differentiation and proliferation, and/or assist maintenance of in vivo cellular functions.

Chemical moieties may have one or more positive or negative charges to induce electrostatic interaction with the charged cell membranes. Examples hereof include, but are not limited to, quaternary ammonium (QA), carboxymethyl (CM), and diethylaminoethyl (DEAE). For many cells, the cell membrane would be negatively charged, and electrostatic interactions would be induced for nanofibrous cellulose scaffolds functionalized with positively charged functional moieties, such as QA or DEAE.

Biological moieties may be any type of biological molecule that can secure cell attachment to the nanofibrous cellulose scaffold, including but not limited to, lipid anchors, cell-adhesion molecules (CAMs), antigens, receptors, and antibodies. CAMs include integrins, immunoglobulins, cadherins, and selectins. Biological moieties may also assist cellular differentiation through receptor-ligand interactions, antigen-antibody interactions, protein interactions, and/or immune stimulatory signalling.

### Adherent Cell

In the present context, the term “adherent cells” refers to any cell that requires a surface or artificial substrate, such as a microcarrier, to form an adherent cell culture. Preferably, the adherent cell is derived from a solid tissue.

Adherent culture is to be distinguished from suspension culture in which cells are grown freely floating in suspension.

### Mean Diameter (of Cellulose Nanofiber)

In the present context, the term “mean diameter” refers to the average diameter of cellulose nanofibers in the nanofibrous cellulose scaffold. The mean diameter may be determined from SEM images of the nanofibrous cellulose scaffold. Preferably, the mean diameter is determined from measurement of at least 100 individual nanofibers within the sample, e.g. by use of image analysis software, such as ImageJ.

The mean diameter of the cellulose nanofibers can be adjusted in the process of preparing the initial cellulose nanofiber material. This may be achieved by varying the parameters of e.g. the electrospinning process.

Preferably, the mean diameter of the cellulose nanofibers in the nanofibrous cellulose scaffold is from about 250 nm to about 750 nm, such as about 400 nm to about 600 nm.

### Mean Length (of Cellulose Nanofiber)

In the present context, the term “mean length” refers to the average length of cellulose nanofibers in the nanofibrous cellulose scaffold. The mean length may be determined as the volume weighted mean value (D[4,3]) measured by light scattering, e.g. on a Malvern Mastersizer S. D[4,3] is also known as the De Brouckere mean value.

The mean fiber length in a sample may be determined using the following settings on a Malvern Mastersizer S:
Range lens: 300RF mm
Presentation: 30HD
Analysis model: Polydisperse
Particle refractive index: (1.5295, 0.1000)
Dispersant refractive index: (1.33000)
Density: 1.5000 g/cm³

Preferably, the mean length of the cellulose nanofibers is from about 30 μm to about 250 μm, such as about 40 μm to about 200 μm, such as about 50 μm to about 150 μm, preferably about 60 μm to about 100 μm.

### Surface Area

In the present context, the term “surface area” refers to the Brunauer-Emmett-Teller (BET) surface area. The BET surface area may be determined by measurement of the physiosorption of a gas, usually nitrogen, to give a value of the sample. The BET method can accurately determine the surface area of the nanofibrous cellulose scaffold since the gas molecules can travel within the nanofibrous matrix to probe also interior surfaces.

The surface area is given as area per unit mass (e.g. cm²/g) and can be measured according to ISO 9277:2022—Determination of the specific surface area of solids by gas adsorption—BET method.

### Degree of Substitution (DS)

In the present context, the term “degree of substitution (DS)” refers to the average number of functional moieties attached per base unit of the condensation polymer cellulose. The base unit of cellulose is β(1->4) linked D-glucose, which comprise three hydroxyl groups that may be subjected to substitution. Accordingly, the theoretical maximum value of DS is 3.

Degree of substitution (DS) may be determined using the following formula:

DS=(162N/(1400−CA×N))

where 162 is the molecular weight of the anhydrous glucose unit (AGU); N is the percentage of nitrogen; CA is the molecular weight of the cationic reagent.

Substitution of the cellulose nanofibers can also be quantified as equivalent of charge per base unit mass of cellulose, and is given in units of meq/g. This value can be determined by zeta potential measurements, pH titration or electrokinetic chromatography.

### Dead Volume

In the present context, the term “dead volume” refers to the volume occupied by the microcarrier when culturing cells. Ideally, the dead volume is minimised to allow for more cell proliferation per volume in the container used for culturing cells.

### Mercerization

In the present context, the term “mercerization” refers to a process comprising swelling of the cellulose nanofiber material in an aqueous or ethanolic NaOH solution to break internal hydrogen bonds of cellulose and increase the number of available hydroxyl groups (—OH).

### Microcarrier

In the present context, the term “microcarrier” refers to any support matrix upon which adherent cells may grow in adherent culture.

### Suspension Based Bioreactor

In the present context, the term “suspension based bioreactor” refers to a bioreactor wherein the microcarriers and cells adhere thereto are freely floating in the bioreactor. In a suspension based bioreactor, it is possible to add more microcarriers and/or medium during culturing.

Thus, the term “suspension based bioreactor” includes, but is not limited to, stirred tank bioreactors, fluidized bed bioreactors, and airlift bioreactors.

In contrast, a suspension based bioreactor is to be distinguished from a bioreactor wherein the microcarriers are fixed within the bioreactor, such as a packed bed bioreactor wherein the microcarriers are immobilised in a bed.

### About

Wherever the term “about” is employed herein in the context of amounts, for example absolute amounts, such as numbers, purities, weights, sizes, etc., or relative amounts (e.g. percentages, equivalents or ratios), timeframes, and parameters such as temperatures, pressure, etc., it will be appreciated that such variables are approximate and as such may vary by ±10%, for example t 5% and preferably t 2% (e.g. t 1%) from the actual numbers specified. This is the case even if such numbers are presented as percentages in the first place (for example ‘about 10%’ may mean±10% about the number 10, which is anything between 9% and 11%).

### Nanofibrous Cellulose Scaffold

Herein are described a nanofibrous cellulose scaffold that may be used as a microcarrier for culturing cells. The nanofibrous cellulose scaffold has properties, such as a large surface area and low volume, which unlocks a significant increase in cell proliferation per unit culturing volume. Importantly, the nanofibrous cellulose scaffold can be produced by a simple and cost-effective method yielding a commercially attractive and readily scalable end-product.

Thus, an aspect of the present invention relates to a method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) functionalizing the processed cellulose nanofiber material by addition of a reagent comprising a functional moiety, and
(iv) drying the processed cellulose nanofiber material,
   thereby providing the nanofibrous cellulose scaffold.

The resulting nanofibrous cellulose scaffold mimics the extracellular matrix (ECM) and has a high surface area due to the homogeneous distribution of cellulose nanofibers. The ECM is important for survival, proliferation, differentiation and migration of the cells, and microcarriers mimicking the properties of the ECM is therefore considered a step in the direction of in vivo-like cell culturing.

The flexible nature of the cellulose nanofibers allows them to form a network of strands that efficiently exploit the space occupied and reduces the microcarrier dead volume that is not accessible to cells. Accordingly, more surface area can be packed into a smaller volume which is advantageous for use in cell culturing containers wherein only a finite volume is available.

The functional moieties substituted onto the nanofibrous cellulose scaffold may include ECM proteins, peptides, and/or charged groups to e.g. increase attachment levels of the cells to the scaffold, promote differentiation of the cells, or assist in the release and isolation of the cells from the scaffold.

The present method therefore provides an improved microcarrier material that may be used for increasing cell growth and product yield.

Dividing of the initial cellulose nanofiber material is important as it ensures a homogeneous distribution of the cellulose nanofibers in the final scaffold. The shorter cellulose nanofibers also significantly reduce the risk of entanglement of the nanofibers and formation of clusters of nanofibers, which leads to less available surface area exposed to the cells and increased risk of blocking the impeller of a bioreactor. Moreover, larger entanglements or clusters of cellulose nanofibers also render the final nanofibrous cellulose scaffold difficult to handle and may cause clogging of tubing or during pipetting. As part of the upstream biomanufacturing process, pipetting or tapping from the bioreactor is continuously performed in order to perform cell counting, viability and yield results. Therefore, a microcarrier solution that has even a small risk of clogging will not be viable.

It has been found that dispersing the initial cellulose nanofiber material results in a homogeneous material that is suitable for use as a microcarrier. Without being bound by theory, it is contemplated that dispersing the initial cellulose nanofiber material results in a distribution which are spaced so as to allow multiple attachment points for the cells leading to improved proliferation.

Accordingly, an embodiment of the present invention relates to the method as described herein, wherein dividing said initial cellulose nanofiber material is achieved by dispersing.

Another embodiment of the present invention relates to the method as described herein, wherein dispersing is performed with a high-speed disperser.

A further embodiment of the present invention relates to the method as described herein, wherein dividing said initial nanofiber material comprises a step of cutting the initial nanofiber material with a disperser.

Increasing the dispersing time reduces the mean length of the cellulose nanofibers in the nanofibrous cellulose scaffold. In particular, it is advantageous to disperse the material for at least a couple of minutes to reduce nanofiber entanglement and cluster formation. Also, without being bound by theory, it is contemplated that longer cellulose nanofibers are not as easily substituted on the hydroxyl groups, thereby leading to a lower degree of substitution (DS) of functional moieties.

Thus, an embodiment of the present invention relates to the method as described herein, wherein dispersing is performed for at least 2 min, such as at least 5 min, such as at least 10 min.

Another embodiment of the present invention relates to the method as described herein, wherein dispersing is performed for at least 2 min, such as at least 5 min, such as at least 10 min, such as at least 15 min, such as at least 20 min, such as at least 30 min, such as at least 40 min, such as at least 50 min, such as at least 60 min, such as at least 90 min, such as at least 120 min.

A further embodiment of the present invention relates to the method as described herein, wherein dispersing is performed for a period of time in the range of 2 min to 120 min, such as 2 min to 90 min, such as 5 min to 60 min, such as 10 min to 60 min, such as 15 min to 60 min.

A preferred embodiment of the present invention relates to the method as described herein, wherein dispersing is performed for at least 5 min.

Another preferred embodiment of the present invention relates to the method as described herein, wherein dispersing is performed for at least 15 min.

A still further preferred embodiment of the present invention relates to the method as described herein, wherein dispersing is performed for at least 60 min.

Dispersing of the initial cellulose nanofiber material for at least 15 min, and even at least 60 min, can be advantageous as it reduces entanglement and cluster formation of the cellulose nanofibers.

Preferably dispersing is performed at high speed, such as at about 18000 rpm. The speed may be adjusted depending on the type of disperser. Any type of disperser may be used, including, but not limited to, laboratory high-speed disperser, pilot-scale high-speed dispersers, and production-scale high-speed dispersers. The speed may be adjusted to produce a good vortex in the solution, and can depend on volume and viscosity of the solution.

Therefore, an embodiment of the present invention relates to the method as described herein, wherein dispersing is performed at a speed in the range of about 10000 rpm to about 30000 rpm, such as at about 12000 rpm to about 25000 rpm, such as about 15000 rpm to about 20000 rpm.

Yet another embodiment of the present invention relates to the method as described herein, wherein dispersing is performed at at least 15000 rpm, such as at at least 20000 rpm, such as at least 25000 rpm.

During the dividing step, the cellulose nanofibers are reduced in length. It is important that the cellulose nanofibers are not too long as it will cause entanglement of the nanofibers and cluster formation. Cells are not able to penetrate and migrate into these tight clusters of entangled fibers and thereby a portion of the large surface area of the nanofibers are lost.

Therefore, an embodiment of the present invention relates to the method as described herein, wherein the mean length of the cellulose nanofibers in said processed cellulose nanofiber material is shorter than the mean length of the cellulose nanofibers in said initial cellulose nanofiber material.

Another embodiment of the present invention relates to the method as described herein, wherein the mean length of the cellulose nanofibers in said processed cellulose nanofiber material is less than 250 μm, such as less than about 200 μm, such as less than about 150 μm, such as less than about 120 μm, such as less than about 100 μm, such as less than about 80 μm.

A further embodiment of the present invention relates to the method as described herein, wherein the mean length of the cellulose nanofibers in said processed cellulose nanofiber material is in the range of about 30 μm to about 250 μm, such as about 40 μm to about 200 μm, such as about 50 μm to about 150 μm, preferably about 60 μm to about 100 μm.

A still further embodiment of the present invention relates to the method as described herein, wherein the mean length of the cellulose nanofibers in said processed cellulose nanofiber material is in the range of about 70 μm to about 120 μm.

The dividing step may be performed under cooling to lower the ductility of the cellulose nanofibers, making them more brittle and easier to divide. The cooling may occur before or during the dividing of the initial cellulose nanofiber material. Cooling may comprise cooling of the container in which the initial cellulose nanofiber material is held during the dividing step or cooling the initial cellulose nanofiber material by exposure to a coolant, such as liquid hydrogen, liquid helium and/or liquid nitrogen, or by keeping the initial cellulose nanofiber material in a fridge or freezer immediately before the dividing step.

Thus, an embodiment of the present invention relates to the method as described herein, wherein the initial cellulose nanofiber material is cooled before or during the dividing step.

Cellulose sheets, if prepared by electrospinning, may be highly static and difficult to handle. Therefore, the initial cellulose nanofiber material may conveniently be provided as a liquid sample that is ready for processing, e.g. by dispersing.

Thus, an embodiment of the present invention relates to the method as described herein, wherein the initial cellulose nanofiber material is provided as a liquid sample.

Another embodiment of the present invention relates to the method as described herein, wherein the solvent of the liquid sample comprises water and/or ethanol.

A further embodiment of the present invention relates to the method as described herein, wherein the solvent of the liquid sample comprises ethanol.

Yet another embodiment of the present invention relates to the method as described herein, wherein the concentration of cellulose nanofibers in the processed cellulose nanofiber material is about 0.1 wt % to about 10 wt %, such as about 0.5 wt % to about 5 wt %, such as about 0.75 wt % to about 4 wt %, preferably about 1 wt % to about 3 wt %, with respect to the total weight of the liquid sample.

The mean diameter of the cellulose nanofibers can be guided during the preparation of the initial cellulose nanofiber material. This may be achieved by varying the parameters of e.g. the electrospinning or meltblowing process, such as the voltage or heat applied, the speed and type of injection, and/or the rotational speed of the collector drum. The mean diameter of the cellulose nanofibers may be varied depending on the application, e.g. the type of cells to be cultured. It has been found that for many applications, a mean diameter of about 400 nm to about 600 nm, such as about 500 nm, is advantageous.

Accordingly, an embodiment of the present invention relates to the method as described herein, wherein the initial cellulose nanofiber material is prepared by a process selected from the group consisting of electrospinning, meltblowing, drawing, self-assembly, template synthesis, and thermal-induced phase separation.

Another embodiment of the present invention relates to the method as described herein, wherein the initial cellulose nanofiber material is prepared by electrospinning.

A further embodiment of the present invention relates to the method as described herein, wherein the mean diameter of the cellulose nanofibers in said initial cellulose nanofiber material and/or processed cellulose nanofiber material is in the range of about 10 nm to about 2000 nm, such as about 50 nm to about 1500 nm, such as about 100 nm to about 1000 nm, preferably about 250 nm to about 750 nm.

In a preferred embodiment of the present invention relates to the to the method as described herein, wherein the mean diameter of the cellulose nanofibers in said initial cellulose nanofiber material and/or processed cellulose nanofiber material is in the range of about 400 nm to about 600 nm, preferably about 500 nm.

The initial cellulose nanofiber material may be prepared from a cellulose acetate solution, e.g. by electrospinning of a cellulose acetate solution. However, the resulting cellulose acetate sheets are preferably regenerated to cellulose sheets in a sodium hydroxide bath before any further processing. This treatment opens up hydroxyl groups that may subsequently be used for binding of functional moieties.

Thus, an embodiment of the present invention relates to the method as described herein, wherein the initial cellulose nanofiber material is prepared by electrospinning of a cellulose acetate solution to cellulose acetate sheets.

Another embodiment of the present invention relates to the method as described herein, wherein the cellulose acetate sheets are regenerated to cellulose sheets by treatment with NaOH.

A still further embodiment of the present invention relates to the method as described herein, wherein said initial cellulose nanofiber material is regenerated in a regeneration solution comprising sodium hydroxide.

Another embodiment of the present invention relates to the method as described herein, wherein the regeneration solution comprises sodium hydroxide in the range of about 0.1M to about 1 M, such as about 0.2 M to about 0.8 M, such as about 0.3 M to about 0.7 M, such as about 0.4 M to about 0.6 M, preferably about 0.5 M.

Regeneration of cellulose sheets is preferably performed in an ethanol solution comprising sodium hydroxide. It is possible to use varying amounts of ethanol, such as from 5% vol/vol to 99% vol/vol.

Thus, an embodiment of the present invention relates to the method as described herein, wherein the regeneration solution is an ethanol solution comprising about 5% vol/vol to about 99% vol/vol ethanol, such as about 10% vol/vol to about 95% vol/vol ethanol, such as about 20% vol/vol to about 90% vol/vol ethanol, such as about 30% vol/vol to about 80% vol/vol ethanol, such as about 40% vol/vol to about 70% vol/vol ethanol.

The content of ethanol in the regeneration solution may influence the mechanical properties of the resulting nanofibrous cellulose scaffold. Mechanical properties that can be impacted by the content of ethanol includes elasticity and brittleness. Without being bound by theory, it is contemplated that a more elastic and less stiff material is beneficial for interaction with the cells and to promote proliferation.

Thus, an embodiment of the present invention relates to the method as described herein, wherein the regeneration solution is an ethanol solution comprising at least about 50% vol/vol ethanol, such as at least 60% vol/vol ethanol, such as at least 70% vol/vol ethanol, such as at least 80% vol/vol ethanol, such as at least 90% vol/vol ethanol, preferably about 90% vol/vol ethanol.

The cellulose nanofibers may be combined with other types of nanofibers to enhance the nanofibrous cellulose scaffold with new properties. This may be beneficial for specific cell types wherein the addition of other types of nanofibers widens the options for mimicking the local extracellular matrix of that particular tissue wherefrom the cells are. The additional polymers may be of natural or synthetic origin. For natural nanofibers, especially types of nanofibers traditionally present in the extracellular environment, such as collagen, may work in synergy with cellulose to mimic the ECM.

Therefore, an embodiment of the present invention relates to the method as described herein, wherein the initial cellulose nanofiber material further comprises one or more nanofibers selected from natural polymers or synthetic polymers.

Another embodiment of the present invention relates to the method as described herein, wherein the natural polymers are selected from the group consisting of collagen, silk fibroin, keratin, gelatin and polysaccharides, and combinations thereof.

A further embodiment of the present invention relates to the method as described herein, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA), and combinations thereof.

Subsequent to dividing of the initial cellulose nanofiber material, the resulting processed material is preferably handled to make it ready for functionalization. Part of the treatment can include filtration, washing and re-suspension of the processed cellulose nanofiber material. Filtration ensures that excess water is removed from the processed cellulose nanofiber material. Washing assists in removal of any acetate ions still present after the previous treatment. When re-suspending the processed cellulose nanofiber material, the concentration of cellulose nanofibers may be adjusted if desired.

Therefore, an embodiment of the present invention relates to the method as described herein, wherein the dividing step (ii) is followed by a step comprising filtration, washing and suspending the processed cellulose nanofiber material.

Another embodiment of the present invention relates to the method as described herein, wherein said filtration comprises sieving of the processed cellulose nanofiber material. Yet another embodiment of the present invention relates to the method as described herein, wherein the suspension comprises water and/or ethanol.

Prior to functionalization, the cellulose nanofibers are preferably mercerized to improve the substitution of functional moieties onto the cellulose nanofibers. Mercerization is a process in which the cellulose nanofibers are swollen by immersion in a concentrated aqueous NaOH solution, washed with water, and dried. During the mercerization process, the crystal structure of the cellulose nanofiber is transformed from cellulose I to cellulose II. Under the action of concentrated alkaline solutions chemical, physicochemical and structural modifications of cellulose occur. Upon washing and neutralisation cellulose II is formed. As a result of the penetration of the base into the lattice, internal hydrogen bonds are broken and the number of available hydroxyl groups (—OH) in the cellulose nanofiber is increased. It is therefore contemplated that mercerization improves the degree of substitution (DS). The mercerization step may be performed before or after the dividing step.

Therefore, an embodiment of the present invention relates to the method as described herein further comprising a step of mercerization of said processed cellulose nanofiber material.

Another embodiment of the present invention relates to the method as described herein, wherein the mercerization step is immediately before or after the dividing step (ii).

Yet another embodiment of the present invention relates to the method as described herein, wherein said mercerization step comprises addition of NaOH.

A further embodiment of the present invention relates to the method as described herein, wherein concentration of NaOH is in the range of about 0.05 M to about 2 M, such as about 0.1 M to about 1.5 M, such as about 0.25 M to about 0.75 M, preferably about 0.4 M to about 0.6 M.

A still further embodiment of the present invention relates to the method as described herein, wherein the mercerization step is performed for a period of about 1 hour to about 3 hours, preferably about 2 hours.

An even further embodiment of the present invention relates to the method as described herein, wherein the degree of substitution of the functional moiety (DS) is in the range of about 0.01 to about 2.

Another embodiment of the present invention relates to the method as described herein, wherein the degree of substitution of the functional moiety (DS) is in the range of about 0.01 to about 1.5, such as about 0.05 to about 1.2, such as about 0.1 to about 1, such as about 0.2 to about 0.8, such as about 0.4 to about 0.6.

The amount of substitution may also be quantified as equivalents of charge per base unit mass of cellulose (meq/g). For positively charged functional moieties, such as QA and DEAE, it has been found that a range of 0.5 to 3.5 meq/g is advantageous.

Thus, an embodiment of the present invention relates to the method as described herein, wherein the functional moiety is QA and/or DEAE, and wherein the equivalents of charge per base unit mass of cellulose on the nanofibrous cellulose scaffold is in the range of about 0.5 meq/g to about 3.5 meq/g, such as 1 meq/g to about 2 meq/g, preferably in the range of about 1.25 meq/g to about 1.75 meq/g.

Functionalization of the nanofibrous cellulose scaffold may also be quantified in terms of the ion exchange capacity of the microcarrier. The ion exchange capacity can be defined as the ability of the functional moiety coupled to the cellulose nanofibers to undergo displacement of ions which are attached to its structure by opposite charged ions available in the surrounding solution. The ion exchange capacity is given in units of mmol Cl⁻/g and is determined via titration.

Therefore, an embodiment of the present invention relates to the method as described herein, wherein the ion exchange capacity of the nanofibrous cellulose scaffold is in the range of about 0.1 mmol Cl⁻/g to about 1.5 mmol Cl⁻/g, such as about 0.3 mmol Cl⁻/g to about 1 mmol Cl⁻/g.

Another embodiment of the present invention relates to the method as described herein, wherein the ion exchange capacity of the nanofibrous cellulose scaffold is at least about 0.1 mmol Cl⁻/g, such as at least about 0.2 mmol Cl⁻/g, preferably at least about 0.3 mmol Cl⁻/g.

A further embodiment of the present invention relates to the method as described herein, wherein the functional moiety is QA and/or DEAE, and wherein the ion exchange capacity of the nanofibrous cellulose scaffold is at least about 0.3 mmol Cl⁻/g.

The functional moieties attached to the cellulose nanofibers may be of either chemical or biological origin. In particular, positively charged groups are advantageous as they induce electrostatic interactions between the nanofibrous cellulose scaffold and cells with a negatively charged membrane, thereby increasing attachment of cells to the microcarrier. Biological moieties include proteins and peptides that are normally an integral part of the interaction between the cell and the extracellular environment. This interaction may further promote attachment of cells to the nanofibrous cellulose scaffold.

Therefore, an embodiment of the present invention relates to the method as described herein, wherein the functional moiety is selected from chemical moieties or biological molecules.

Another embodiment of the present invention relates to the method as described herein, wherein the chemical moieties are selected from the group consisting of negatively charged groups, positively charged groups, zwitterionic groups, hydrophobic groups, hydrophilic groups, amphiphilic groups, ligands, and combinations thereof.

A further embodiment of the present invention relates to the method as described herein, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

A preferred embodiment of the present invention relates to the method as described herein, wherein the chemical moiety is quaternary ammonium (QA).

It is to be understood that the chemical moieties may be attached to the cellulose backbone using conventional chemistry. Thus reagents such as, but not limited to, 3-chloro-2-hydroxypropyl trimethyl ammonium chloride (CHPTAC), 2-chloro-N,N diethylethylamine hydrochloride (DAECH), and monochloro acetic acid (MCAA) may be used for attachment of QA, DEAE, and CM, respectively.

An even further embodiment of the present invention relates to the method as described herein, wherein the biological molecules are selected from the group consisting of proteins, peptides, antibodies, amino acids, polypeptides, glycoproteins, lipoproteins, and antigens, and combinations thereof.

After functionalization of the processed cellulose nanofiber scaffold, the material is dried to yield the nanofibrous cellulose scaffold in its final form. Drying may be performed in two steps, such as freezing followed by lyophilization, or in a single step, such as by lyophilization. A lyophilizer performs a water removal process that can extend shelf life and/or make the material more convenient for transport. Lyophilizers work by freezing the material, then reducing the pressure and adding heat to allow the frozen water in the material to sublimate.

Accordingly, an embodiment of the present invention relates to the method as described herein, wherein the drying step (iv) comprises freezing and/or lyophilization of the processed cellulose nanofiber material.

Another embodiment of the present invention relates to the method as described herein, wherein the drying step (iv) comprises freezing of processed cellulose nanofiber material followed by lyophilization of the frozen processed cellulose nanofiber material.

The dried nanofibrous cellulose scaffold may be further processed to provide a dry powder. This may be accomplished by grinding the dried product.

The method described herein provides a nanofibrous cellulose scaffold with large surface area and low dead volume that may advantageously be utilised as a microcarrier for culturing of cells. The microcarrier product may be in the form of a dry powder that is added to the cell culturing container, such as a bioreactor, to form a support matrix upon which cells may attach and proliferate.

Thus, an aspect of the present invention relates to a nanofibrous cellulose scaffold obtainable from a method as described herein.

Another aspect of the present invention relates to a nanofibrous cellulose scaffold comprising a processed cellulose nanofiber material with a mean length of the cellulose nanofibers of less than about 250 μm, and wherein the processed cellulose nanofiber material is functionalized with a functional moiety.

The nanofibrous cellulose scaffold has a high surface area. Without being bound by theory, it is contemplated that the high surface area, amongst others, is caused by the homogenous distribution of processed cellulose nanofibers in the material, which ensures that large entanglements and clusters of nanofibers are avoided. Accordingly, the nanofibrous cellulose scaffold provides a superior available surface area that is advantageous for culturing of adherent cells.

An embodiment of the present invention relates to the nanofibrous cellulose scaffold as described herein, wherein the nanofibrous cellulose scaffold has a BET surface area of at least about 40000 cm²/g, such as at least about 50000 cm²/g, such as at least about 55000 cm²/g, such as at least about 60000 cm²/g, such as at least about 70000 cm²/g, such as at least about 80000 cm²/g.

Another embodiment of the present invention relates to the nanofibrous cellulose scaffold as described herein, wherein the nanofibrous cellulose scaffold has a BET surface area of at least about 25000 cm²/g, such as at least about 30000 cm²/g, such as at least about 40000 cm²/g, such as at least about 50000 cm²/g, such as at least about 60000 cm²/g, such as at least about 70000 cm²/g.

Yet another embodiment of the present invention relates to the nanofibrous cellulose scaffold as described herein, wherein BET surface area is measured according to ISO 9277:2022—Determination of the specific surface area of solids by gas adsorption—BET method.

Preferably, the cellulose nanofibrous scaffold is provided as a dry material that is convenient and easy to handle for the end user. The dry material may be packed in a container suitable for direct addition to the cell culturing container. The microcarrier in dry powder form may also be reconstituted/rehydrated in aqueous solution, such as PBS and/or culture medium, prior to addition to the cell culturing container.

Thus, an embodiment of the present invention relates to the nanofibrous cellulose scaffold as described herein, wherein the nanofibrous cellulose scaffold is provided as a dry material.

Another embodiment of the present invention relates to the nanofibrous cellulose scaffold as described herein, wherein the nanofibrous cellulose scaffold is provided as a lyophilized material.

Despite the fact that bacterial, yeast, and insect cell expressions systems are capable of overexpressing recombinant proteins, culturing of mammalian cells remains a cornerstone for biomanufacturing of biologics because of their ability to propagate human viruses, express monoclonal antibodies, and incorporate post-translational modifications, such as glycosylation that are critical to the production of effective biologics. Among the most utilised mammalian cell lines are human embryonic kidney (HEK) 293 cells that have been engineered to produce therapeutic proteins and antibodies at high levels. However, HEK 293 cells and other adherent mammalian cells are highly dependent on a suitable support matrix for cell proliferation and viability. Thus, microcarriers are important products to continue pushing biomanufacturing to become more cost-effective.

The nanofibrous cellulose scaffold described herein offers all the traits (high surface area, low dead volume, customizable) that are necessary for an efficient microcarrier.

Importantly, the nanofibrous cellulose scaffold can easily be scaled for industrial use without cost and applicability of the microcarrier when used at high volumes being a hindrance.

Accordingly, an aspect of the present invention relates to a microcarrier comprising a nanofibrous cellulose scaffold as described herein.

Another aspect of the present invention relates to use of a nanofibrous cellulose scaffold as described herein as a microcarrier for cell culturing.

A further aspect of the present invention relates to a cell culturing device comprising a container loaded with a nanofibrous cellulose scaffold or a microcarrier as described herein.

The container of the cell culturing devices is not limited to any particular container as long as it is a suitable vessel for culturing of cells.

Therefore, an embodiment of the present invention relates to the cell culturing device as described herein, wherein the container is selected from the group consisting of a bioreactor, a cell culturing plate, a cell culture bottle, a spinner flask, a shaker flask, and a roller bottle, a petri dish, and a tube, preferably a bioreactor.

Bioreactors are of particular importance to large scale industrial production. They may hold larger volumes than laboratory- or pilot study equipment and are used in upstream processes to expand and scale cell culture for production. A typical process involves initial expansion of cells in a smaller vessel followed by successive expansion into larger culture vessels. When the culture volume and density is optimal, the cells are transferred to a production bioreactor, which offers a controlled microenvironment and nutrient delivery to regulate cell growth and differentiation, improving standardization and reproducibility. The nanofibrous cellulose scaffold is suitable for use at any point in this process, and is particularly advantageous at the production scale where other microcarriers may be too expensive or otherwise unsuitable for use.

Thus, an embodiment of the present invention relates to the cell culturing device as described herein, wherein the container is a bioreactor, preferably a suspension based bioreactor.

The nanofibrous cellulose scaffold is suitable for use all the way from lab scale (1 ml to 5 L), via pilot scale/product development (1 L to 100 L), and to production scale (100 L to 1000 L). If required, the material may be also used as microcarrier in even larger bioreactors.

Thus, an embodiment of the present invention relates to the cell culturing device as described herein, wherein the volume of the container is in the range of about 1 mL to about 1000 L, such as about 1 L to about 1000 L, such as about 100 L to about 1000 L.

Another embodiment of the present invention relates to the cell culturing device as described herein, wherein the volume of the container is at least 10 L, such as at least 50 L, such as at least 100 L, such as at least 200 L, such as at least 500 L, such as at least 1000 L.

The container is not limited to any particular material but may be from e.g. stainless steel or of a disposable material, the latter of which provides flexibility and reduces downtime caused by the need for cleaning and sterilization of the culturing device.

Another embodiment of the present invention relates to the cell culturing device as described herein, wherein the cell culturing device comprises a solvent.

A further embodiment of the present invention relates to the cell culturing device as described herein, wherein the solvent is cell culturing medium.

The nanofibrous cellulose scaffold may be utilised as microcarrier in a conventional method for cell culturing. The nanofibrous cellulose scaffold may be provided as part of a cell culturing device comprising a container loaded with the microcarrier. It is contemplated that the nanofibrous cellulose scaffold is suitable for use in culturing of any cell line that may benefit from interaction with a support matrix during culturing. It is to be understood that the nanofibrous cellulose scaffold can be used in any traditional scale up step for cell culturing, i.e. first adding microcarrier and cells to a small container (e.g. a flask), then move the cell population to a larger container, before finally transferring the cell culture on to a bioreactor.

Thus, an aspect of the present invention relates to a method for cell culturing comprising the steps of:
(i) providing a cell culturing device as described herein,
(ii) adding a composition comprising a cell population to the cell culturing device,
(iii) incubating the cell population to provide a proliferated cell population, and
(iv) optionally, extracting the proliferated cell population from the cell culturing device.

An embodiment of the present invention relates to the method for cell culturing as described herein, wherein the composition comprises a solvent.

Another embodiment of the present invention relates to the method for cell culturing as described herein, wherein the solvent comprises a cell culturing medium.

The method for cell culturing is particular advantageous for culturing of adherent cells which grow while adhering to the culture vessel. Ideally, cultured cells are cultured in a manner that reflects the conditions under which they exist in the living organism. Adherent cells are cells which under native conditions adhere to tissues.

Thus, an embodiment of the present invention relates to the method for cell culturing as described herein, wherein the cell population comprises adherent cells.

Another embodiment of the present invention relates to the method for cell culturing as described herein, wherein the origin of the cell population is selected from the group consisting of mammalian cells, human cells, yeast cells, bacterial cells, insect cells and plant cells, preferably mammalian cells.

A further embodiment of the present invention relates to the method for cell culturing as described herein, wherein the cell population comprises a cell type selected from the group consisting of stem cells, kidney cells, lung cells, liver cells, pancreas cells, heart cells, ovary cells, hybridoma cells, and immortalised cells.

A still further embodiment of the present invention relates to the method for cell culturing as described herein, wherein the cell population comprises a cell type selected from the group consisting of myocytes, myoblasts, and adipocytes.

For the purpose of biomanufacturing, some cell lines are preferred due to characteristics such as ease of handling, ability to propagate human viruses, or implementation of favourable glycosylation patterns. An example hereof is HEK293 cells, which may be used for packaging and amplification of recombinant adenovirus.

Therefore, an embodiment of the present invention relates to the method for cell culturing as described herein, wherein the cell population comprises a cell line selected from the group consisting of HEK293 cells, ARN8 cells, Vero cells, MRC-5 cells, WI-38 cells, CHO cells, BHK-21 cells, Sf9 cells, mesenchymal stem cells, pluripotent stem cells, and MDCK cells.

A further embodiment of the present invention relates to the method for cell culturing as described herein, wherein the cell population comprises stem cells.

Another embodiment of the present invention relates to the method for cell culturing as described herein, wherein the cell population comprises a HEK 293 cell line.

The method for cell culturing may also readily be used for cellular agriculture products and new ways of producing existing agricultural products like milk, and (cultured) meat from cells. Cellular agriculture is considered a means of achieving animal-free agriculture. As cellulose is a biocompatible polymer, it is contemplated herein that the nanofibrous cellulose scaffold may be used for cellular agriculture.

The method for culturing cells can be performed with or without agitation of the cell culture within the container. If present, agitation is preferably effected by stirring of the medium in which the cells grow. Agitation promotes better transport of nutrients and oxygen to the cells.

Therefore, an embodiment of the present invention relates to the method for cell culturing as described herein, wherein incubation is performed without agitation.

Another embodiment of the present invention relates to the method for cell culturing as described herein, wherein incubation is performed under agitation.

A further embodiment of the present invention relates to the method for cell culturing as described herein, wherein agitation is selected from the group consisting of stirring, shaking, rocking, waving, and bubbling, preferably stirring.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the method for preparing the nanofibrous cellulose scaffold and all its features, which may readily be part of the nanofibrous cellulose scaffold per se, or a use or method using the same for cell culturing. Embodiments and features of the present invention are also outlined in the following items.

### Items

X1. A method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) functionalizing the processed cellulose nanofiber material by addition of a reagent comprising a functional moiety, and
(iv) drying the processed cellulose nanofiber material, thereby providing the nanofibrous cellulose scaffold.

X2. The method according to item X1, wherein dividing said initial cellulose nanofiber material is achieved by dispersing.

X3. The method according to item X2, wherein dispersing is performed with a high-speed disperser.

X4. The method according to any one of items X2 or X3, wherein dispersing is performed for at least 2 min, such as at least 5 min, such as at least 10 min.

X5. The method according to any one of items X2-X4, wherein dispersing is performed at a speed in the range of about 10000 rpm to about 30000 rpm, such as at about 12000 rpm to about 25000 rpm, such as about 15000 rpm to about 20000 rpm.

X6. The method according to any one of the preceding items, wherein the mean length of the cellulose nanofibers in said processed cellulose nanofiber material is shorter than the mean length of the cellulose nanofibers in said initial cellulose nanofiber material.

X7. The method according to any one of the preceding items, wherein the mean length of the cellulose nanofibers in said processed cellulose nanofiber material is less than 250 μm, such as less than about 200 μm, such as less than about 150 μm, such as less than about 120 μm, such as less than about 100 μm, such as less than about 80 μm.

X8. The method according to any one of the preceding items, wherein the mean length of the cellulose nanofibers in said processed cellulose nanofiber material is in the range of about 30 μm to about 250 μm, such as about 40 μm to about 200 μm, such as about 50 μm to about 150 μm, preferably about 60 μm to about 100 μm.

X9. The method according to any one of the preceding items, wherein the initial cellulose nanofiber material is provided as a liquid sample.

X10. The method according to item X9, wherein the solvent of the liquid sample comprises water and/or ethanol.

X11. The method according to any one of the preceding items, wherein the concentration of cellulose nanofibers in the processed cellulose nanofiber material is about 0.1 wt % to about 10 wt %, such as about 0.5 wt % to about 5 wt %, such as about 0.75 wt % to about 4 wt %, preferably about 1 wt % to about 3 wt %, with respect to the total weight of the liquid sample.

X12. The method according to any one of the preceding items, wherein the mean diameter of the cellulose nanofibers in said initial cellulose nanofiber material and/or processed cellulose nanofiber material is in the range of about 10 nm to about 2000 nm, such as about 50 nm to about 1500 nm, such as about 100 nm to about 1000 nm, preferably about 250 nm to about 750 nm.

X13. The method according to any one of the preceding items, wherein the initial cellulose nanofiber material further comprises one or more nanofibers selected from natural polymers or synthetic polymers.

X14. The method according to item X13, wherein the natural polymers are selected from the group consisting of collagen, silk fibroin, keratin, gelatin and polysaccharides, and combinations thereof.

X15. The method according to any one of items X13 or X14, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA), and combinations thereof.

X16. The method according to any one of the preceding items, wherein the initial cellulose nanofiber material is prepared by a process selected from the group consisting of electrospinning, meltblowing, drawing, self-assembly, template synthesis, and thermal-induced phase separation.

X17. The method according to any one of the preceding items further comprising a step of mercerization of said processed cellulose nanofiber material.

X18. The method according to item X17, wherein the mercerization step is immediately before or after the dividing step (ii).

X19. The method according to any one of items X17 or X18, wherein said mercerization step comprises addition of NaOH.

X20. The method according to item X19, wherein concentration of NaOH is in the range of about 0.05 M to about 2 M, such as about 0.1 M to about 1.5 M, such as about 0.25 M to about 0.75 M, preferably about 0.4 M to about 0.6 M.

X21. The method according to any one of items X17-X20, wherein the mercerization step is performed for a period of about 1 hour to about 3 hours, preferably about 2 hours.

X22. The method according to any one of the preceding items, wherein the functional moiety is selected from chemical moieties or biological molecules.

X23. The method according to item X22, wherein the chemical moieties are selected from the group consisting of negatively charged groups, positively charged groups, zwitterionic groups, hydrophobic groups, hydrophilic groups, amphiphilic groups, ligands, and combinations thereof.

X24. The method according to any one of items X22 or X23, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

X25. The method according to any one of items X22-X24, wherein the chemical moiety is quaternary ammonium (QA).

X26. The method according to any one of items X22-X25, wherein the biological molecules are selected from the group consisting of proteins, peptides, antibodies, amino acids, polypeptides, glycoproteins, lipoproteins, and antigens, and combinations thereof.

X27. The method according to any one of the preceding items, wherein the degree of substitution of the functional moiety (DS) is in the range of about 0.01 to about 2.

X28. The method according to any one of the preceding items, wherein the dividing step (ii) is followed by a step comprising filtration, washing and suspending the processed cellulose nanofiber material.

X29. The method according to item X28, wherein said filtration comprises sieving of the processed cellulose nanofiber material.

X30. The method according to any one of items X28 or X29, wherein the suspension comprises water and/or ethanol.

X31. The method according to any one of the preceding items, wherein the drying step (iv) comprises freezing and/or lyophilization of the processed cellulose nanofiber material.

X32. The method according to any one of the preceding items, wherein the drying step (iv) comprises freezing of processed cellulose nanofiber material followed by lyophilization of the frozen processed cellulose nanofiber material.

Z1. A nanofibrous cellulose scaffold obtainable from a method according to any one of items X1-X32.

Y1. A nanofibrous cellulose scaffold comprising a processed cellulose nanofiber material with a mean length of the cellulose nanofibers of less than about 250 μm, and wherein the processed cellulose nanofiber material is functionalized with a functional moiety.

Y2. The nanofibrous cellulose scaffold according to item Y1, wherein the cellulose nanofibers have a mean length in the range of about 30 μm to about 250 μm, such as about 40 μm to about 200 μm, such as about 50 μm to about 150 μm, preferably about 60 μm to about 100 μm.

Y3. The nanofibrous cellulose scaffold according to any one of items Y1 or Y2, wherein the cellulose nanofibers have a mean diameter in the range of about 10 nm to about 2000 nm, such as about 50 nm to about 1500 nm, such as about 100 nm to about 1000 nm, preferably about 250 nm to about 750 nm.

Y4. The nanofibrous cellulose scaffold according to any one of items Y1-Y3, wherein the processed cellulose nanofiber material is electrospun, meltblown or drawn, preferably electrospun.

Y5. The nanofibrous cellulose scaffold according to any one of items Y1-Y4, wherein the functional moiety is selected from chemical moieties or biological molecules.

Y6. The nanofibrous cellulose scaffold according to item Y5, wherein the chemical moieties are selected from the group consisting of negatively charged groups, positively charged groups, zwitterionic groups, hydrophobic groups, hydrophilic groups, amphiphilic groups, ligands, and combinations thereof.

Y7. The nanofibrous cellulose scaffold according to any one of items Y5 or Y6, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

Y8. The nanofibrous cellulose scaffold according to any one of items Y5-Y7, wherein the chemical moiety is quaternary ammonium (QA).

Y9. The nanofibrous cellulose scaffold according to any one of items Y5-Y8, wherein the biological molecules are selected from the group consisting of proteins, peptides, antibodies, amino acids, polypeptides, glycoproteins, lipoproteins, and antigens, and combinations thereof.

Y10. The nanofibrous cellulose scaffold according to any one of items Y1-Y9, wherein the degree of substitution of the functional moiety (DS) is in the range of about 0.01 to about 2.

Y11. The nanofibrous cellulose scaffold according to any one of items Y1-Y10, wherein the nanofibrous cellulose scaffold is provided as a dry material.

Y12. The nanofibrous cellulose scaffold according to any one of items Y1-Y11, wherein the nanofibrous cellulose scaffold is provided as a lyophilized material.

Y13. The nanofibrous cellulose scaffold according to any one of items Y1-Y12, wherein the nanofibrous cellulose scaffold further comprises one or more nanofibers selected from natural polymers or synthetic polymers.

Y14. The nanofibrous cellulose scaffold according to item Y13, wherein the natural polymers are selected from the group consisting of collagen, silk fibroin, keratin, gelatin and polysaccharides, and combinations thereof.

Y15. The nanofibrous cellulose scaffold according to any one of items Y13 or Y14, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA), and combinations thereof.

Y16. The nanofibrous cellulose scaffold according to any one of items Y1-Y15, wherein the nanofibrous cellulose scaffold has a BET surface area of at least about 40000 cm²/g, such as at least about 50000 cm²/g, such as at least about 55000 cm²/g, such as at least about 60000 cm²/g, such as at least about 70000 cm²/g, such as at least about 80000 cm²/g.

A1. A microcarrier comprising a nanofibrous cellulose scaffold according to any one of items Y1-Y16 or Z1.

U1. Use of a nanofibrous cellulose scaffold according to any one of items Y1-Y16 or Z1 as a microcarrier for cell culturing.

V1. A cell culturing device comprising a container loaded with a nanofibrous cellulose scaffold according to any one of items Y1-Y16 or Z1 or a microcarrier according to item A1.

V2. The cell culturing device according to item V1, wherein the container is selected from the group consisting of a bioreactor, a cell culturing plate, a cell culture bottle, a spinner flask, a shaker flask, and a roller bottle, a petri dish, and a tube, preferably a bioreactor.

V3. The cell culturing device according to any one of items V1 or V2, wherein the container is a bioreactor, preferably a suspension based bioreactor.

V4. The cell culturing device according to any one of items V1-V3, wherein the volume of the container is at least 10 L, such as at least 50 L, such as at least 100 L, such as at least 200 L, such as at least 500 L, such as at least 1000 L.

V5. The cell culturing device according to any one of items V1-V4, wherein the cell culturing device comprises a solvent.

V6. The cell culturing device according to item V5, wherein the solvent is cell culturing medium.

T1. A method for cell culturing comprising the steps of:
(i) providing a cell culturing device according to any one of items V1-V6,
(ii) adding a composition comprising a cell population to the cell culturing device,
(iii) incubating the cell population to provide a proliferated cell population, and
(iv) optionally, extracting the proliferated cell population from the cell culturing device.

T2. The method according to item T1, wherein the composition comprises a solvent.

T3. The method according to item T2, wherein the solvent comprises a cell culturing medium.

T4. The method according to any one of items T1-T3, wherein the cell population comprises adherent cells.

T5. The method according to any one of items T1-T4, wherein the origin of the cell population is selected from the group consisting of mammalian cells, human cells, yeast cells, bacterial cells, insect cells and plant cells, preferably mammalian cells.

T6. The method according to any one of items T1-T5, wherein the cell population comprises a cell type selected from the group consisting of stem cells, kidney cells, lung cells, liver cells, pancreas cells, heart cells, ovary cells, hybridoma cells, and immortalised cells.

T7. The method according to any one of items T1-T6, wherein the cell population comprises a cell line selected from the group consisting of HEK293 cells, ARN8 cells, Vero cells, MRC-5 cells, WI-38 cells, CHO cells, BHK-21 cells, Sf9 cells, mesenchymal stem cells, pluripotent stem cells, and MDCK cells.

T8. The method according to any one of items T1-T7, wherein incubation is performed without agitation.

T9. The method according to any one of items T1-T8, wherein incubation is performed under agitation.

T10. The method according to item T9, wherein agitation is selected from the group consisting of stirring, shaking, rocking, waving, and bubbling, preferably stirring.

The invention will now be described in further details in the following non-limiting examples.

### EXAMPLES

### Example 1: Preparation of Nanofibrous Cellulose Scaffold

In this example is given a non-limiting demonstration of how the nanofibrous cellulose scaffold can be prepared. The core properties of the material, such as surface area and degree of substitution, was characterized.

### Method

### Preparation of Initial Cellulose Nanofiber Material

A solution of 19% cellulose acetate was prepared by addition of cellulose acetate (6.38 g) to 12 ml of acetone in an Erlenmeyer flask, followed by addition of 12 ml DMF and 6 ml 96% ethanol. The solution was stirred using a magnetic stirrer overnight at room temperature.

The 19% cellulose acetate solution was electrospun to prepare cellulose acetate nanofiber sheets. The cellulose nanofibers were electrospun (Fluidnatek LE50) using a drum speed of 200 rpm and a flow of 10 ml/hour at 18 kV+(emitter) and 10 kV- (collector). The cellulose acetate nanofibers were collected on an aluminium substrate, with temperature being 23° C. and at a relative humidity of 63%. After the electrospinning process completed, the cellulose acetate sheet comprising cellulose nanofibers was removed from the drum.

The cellulose acetate sheets were regenerated to cellulose by submergence in a 0.5 M NaOH solution in ethanol. The cellulose acetate sheets were left in the solution for 6 hours followed by transfer to a sieve and washing with copious volumes of distilled water. The washed sheets were put in the oven at 80° C. for 12 hours to provide dry cellulose sheets. The cellulose sheets were weighed.

### Processing of Initial Cellulose Nanofiber Material

The dry cellulose sheets were cut into rough pieces of approximately 2×2 cm squares using scissors. The size of the pieces does not have to be exact, but larger pieces should be avoided as they may hamper the dividing step. The rough pieces of cellulose were fully submerged in water and dispersed using a high-speed disperser (IKA T25 digital Ultra Turrax) at 18,000 rpm for 60 min (with intermittent stops to cool the disperser). The processed cellulose nanofiber material was transferred to a sieve, washed with water to remove any remaining acetate ions, and drained to remove excess water. Cellulose nanofiber material was transferred to a flask and fresh water was added to provide a cellulose concentration of 2 wt %.

This sample without functionalization yields a nanofibrous cellulose scaffold termed “Cellevate No-Func”.

### Functionalization of Processed Cellulose Nanofiber Material

The processed cellulose nanofiber material was functionalized with different chemical moieties according to the processes below.

### Quaternary Ammonium (QA):

0.4 g NaOH was dissolved in 20 ml water. 0.4 g regenerated cellulose nanofiber material was then added to the solution. Mercerization continued for 2 hours at room temperature.

The temperature of the cellulose suspension was increased to 80° C. and 3.4 ml of 60% 3-chloro-2-hydroxypropyltrimethyl ammonium chloride (CHPTAC) was added dropwise. The reaction was continued at 80° C. for 4 hours. Then the reaction mixture was filtered after cooling down to room temperature and washed repeatedly with water to remove any unreacted CHPTAC or NaOH. Finally, the filtered processed and functionalized cellulose nanofiber material was mixed with water to make a 2% suspension again for further experimentation.

This functionalization protocol yielded a nanofibrous cellulose scaffold termed “Cellevate QA”.

### Carboxymethyl (CM):

0.6 g NaOH was dissolved in 25 ml ethanol. 0.5 g regenerated cellulose nanofiber material was then added to the solution. Mercerization was continued for 1 hour at room temperature.

The temperature of the cellulose suspension was increased to 60° C. and 0.14 g monochloro acetic acid (MCAA) dissolved in 1.2 ml ethanol was added dropwise. The reaction was continued at 60° C. for 2 hours. Filtration and washing were performed as for QA functionalization.

This functionalization protocol yielded a nanofibrous cellulose scaffold termed “Cellevate CMC”.

### Carboxymethyl Diethylaminoethyl (CM-DEAE):

8.7 g diethylamino ethyl chloride hydrochloride (DAECH) was dissolved in 100 ml water. 1 g regenerated cellulose nanofiber material was then added to the solution under stirring. The reaction was carried out over 15 minutes at 40° C.

Subsequently, the regenerated cellulose nanofiber material was transferred to a 0.5 M NaOH solution at 80° C. for 10 min to complete the reaction. Filtration and washing was performed as for QA functionalization.

The DEAE-functionalized cellulose nanofiber material was then subjected to the CM protocol described above to yield a DEAE-CM-functionalized nanofibrous cellulose scaffold termed “Cellevate DEAE+CM”.

### Dryinq

The functionalized or non-functionalized cellulose nanofiber material was transferred to a −85° C. freezer and left overnight. The frozen cellulose nanofiber material was then transferred to a lyophilizer and processed for 48 hours to yield a dry product. The dry product was grinded to provide a dry powder of nanofibrous cellulose scaffold.

### Microscopy

Samples of nanofibrous cellulose scaffold (diluted to 0.2 wt % cellulose) were added to a glass slide. Images of the network of cellulose nanofibers in the sample were captured on a light microscope (Leica) using 40× magnification.

### Scanning Electron Microscopy

Morphological studies of the nanofibrous cellulose scaffolds were carried out using scanning electron microscopy (SEM). The samples were dried and sputter coated with gold before carrying out the analysis. The micrographs were obtained in secondary electron (SE) imaging mode on a Hitachi SU3500 at an accelerating voltage of 5 kV and a working distance of 7 mm at varying magnifications.

### Surface Area Measurements

The Brunauer-Emmett-Teller (BET) model was employed to measure the specific surface areas using a molecule of nitrogen bearing molecular cross-sectional area of 0.162 nm². Dried samples were degassed under vacuum for 6 hours before the BET surface area was measured. Nitrogen adsorption and desorption isotherms were acquired on an ASAP 2020 M analyzer (Micromeritics) at 77.3 K.

### Elemental Analysis and Degree of Substitution

Elemental analysis of the nanofibrous cellulose scaffold was performed on an elemental analyzer FlashEA 1112 (Thermo Fischer Scientific) using 5 mg of sample. The samples were well dried before the analysis to remove any adsorbed moisture.

Degree of substitution (DS) of QA functionalized cellulose (i.e. Cellevate QA) was calculated using the following formula:

DS=(162N/(1400−CA×N))

where 162 is the molecular weight of the anhydrous glucose unit (AGU); N is the percentage of nitrogen; CA is the molecular weight of the cationic reagent. For the present nanofibrous cellulose scaffold the cationic reagent is CHPTAC with a molecular weight of 188.1.

### Results

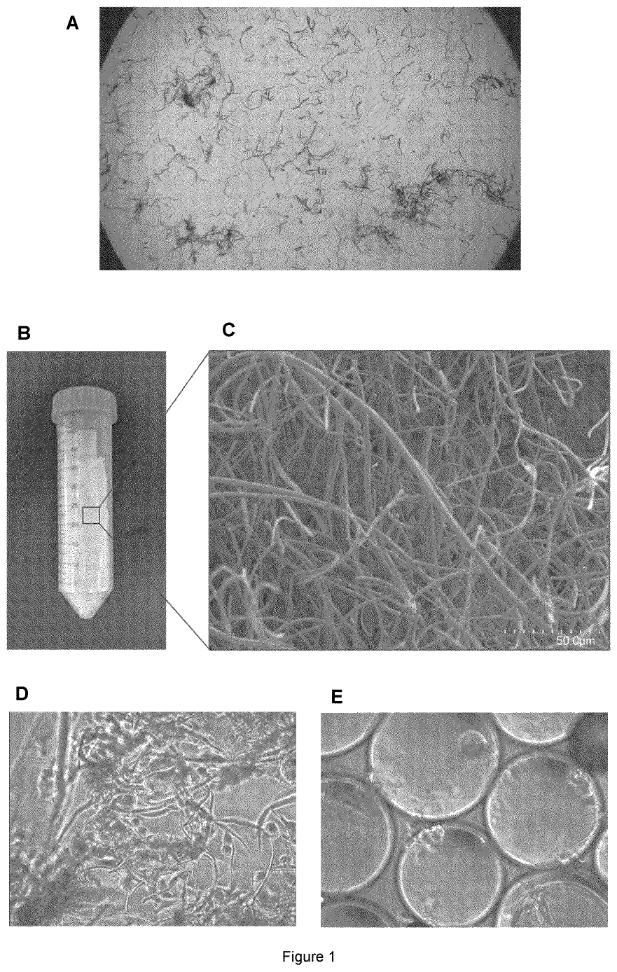

Nanofibrous cellulose scaffolds with different types of functional moieties were prepared and the dry powder product (FIG. 1B) was visualized by light microscopy (FIG. 1A) and SEM (FIG. 1C). The resulting nanofibrous cellulose material is highly homogenous in the sense that individual nanofibers are clearly visible without any large degree of entanglement or clusters. The homogenous distribution of nanofibers in the material ensures optimal exposure of the surface area for cell attachment and interaction.

Elemental composition and degree of substitution (DS) was assessed for the nanofibrous cellulose scaffold functionalized with QA (Cellevate QA). The content of carbon (C) was 37.71%, the content of hydrogen (H) was 6.59%, and the content of nitrogen (N) was 1.28%. Based on the content of N, the DS was calculated to 0.18. The amount of substituted QA may influence the cell growth as the positive charge facilitates electrostatic interactions with negatively charged cell membranes.

In table 1 is listed the nanofibrous cellulose scaffold samples prepared as well as the benchmark commercial microcarrier, Cytodex-1 (Cytiva), which is a spherical dextran particle functionalized with diethylaminoethyl (DEAE).

The surface area of the nanofibrous cellulose scaffold can be approximated in a theoretical calculation using the following equation:

Theoretical surface area per weight (cm²/g)=2/(rΔδ)

wherein m is the mass of nanofibrous cellulose material, r is the radius of the cellulose nanofibers, and δ is the density of cellulose sheets.

The nanofibrous cellulose scaffold offers an increased surface area compared to the market standard microcarrier. The accuracy of the theoretical calculation was supported by a measurement of the BET surface area of a 0.5% cellulose sample, which gave a BET surface area of 58000 cm²/g.

TABLE 1
Summary of the samples compared herein. The surface area of the Cytodex-1
microcarrier is taken from the data sheet of the product, whereas the surface
area of the nanofibrous cellulose scaffolds is a theoretical calculation.
Fiber length and diameter was obtained as described in Example 4.

| | | | Surface area | Material |
|---|---|---|---|---|
| Microcarrier | Functionalization | Material | (cm²/g) | dimensions |
| Cellevate | None | Cellulose | 53333 | Fiber length: 72 μm |
| Non-func. | | | | Fiber diameter: 500 nm |
| Cellevate | Diethylaminoethyl | Cellulose | 53333 | Fiber length: 72 μm |
| DEAE | | | | Fiber diameter: 500 nm |
| Cellevate | Carboxymethyl | Cellulose | 53333 | Fiber length: 72 μm |
| CMC | | | | Fiber diameter: 500 nm |
| Cellevate QA | Quaternary | Cellulose | 53333 | Fiber length: 72 μm |
| | ammonium | | | Fiber diameter: 500 nm |
| Cellevate | Diethylaminoethyl + | Cellulose | 53333 | Fiber length: 72 μm |
| DEAE + CMC | carboxymethyl | | | Fiber diameter: 500 nm |
| Cytodex-1 | Diethylaminoethyl | Dextran | 3000 | Bead diameter: 190 μm |

### Conclusion

This example demonstrates that it is possible to produce several different variants of the nanofibrous cellulose scaffold in a simple manner which is readily scalable for industrial usage. The nanofibrous cellulose scaffold has a large surface area which is available to the cells and does not comprise nanofibers collapsed in clusters or entangled in a fashion that exclude cells from gaining access to the surface.

### Example 2: Cell Proliferation on Nanofibrous Cellulose Scaffold

In this example the ability of cells to grow on the nanofibrous cellulose scaffolds was assessed. To probe properties of the cellulose nanofibers beyond the available surface area, the amount of added microcarrier is adjusted to present the cells with equal amount of surface area, thereby allowing a direct comparison between the nanofibrous cellulose scaffolds and the market standard microcarrier, Cytodex-1.

### Method

### Cell Expansion

HEK293AD and ARN8 cells were seeded at a density of 10,000 cells/cm² (a total number of 1.75×10⁶ cells) in 30 ml Dulbecco's modified eagle medium (DMEM) supplemented with 10% fetal calf serum (FCS) on 175 cm² Nunc flask. After three days, the cell media was removed. The cell monolayer was then washed once with PBS buffer. To detach cells from the flask, 1 ml of Trypsin-EDTA (0.25%) was added to the flask and incubated at 37° C. for at least 1 minute. Once cells detached from the flask, 9 ml of DMEM 10% FCS were added to stop trypsin activity. Cells were suspended and homogenized by pipetting before being counted on a haemocytometer.

### Preparation of Microcarriers

The nanofibrous cellulose scaffolds were prepared as described in example 1. The following samples were assessed; Cellevate No-Func, Cellevate CMC, Cellevate QA, and Cellevate DEAE+CMC.

For this experiment, Cytodex-1 and nanofibrous cellulose scaffold microcarriers were prepared with the same approximated available surface area. Since the surface area per volume differs between the carriers, different amounts of microcarriers were used.

Cytodex-1 microcarrier was prepared in PBS according to manufacturer recommendations. 1.33 g of Cytodex-1 dry beads was prepared by dilution in 40 ml PBS before being autoclaved. The autoclaved beads have a diameter of 190 μm, and have a surface area of 3000 cm²/g. The total surface area per volume was 100 cm²/ml. (1.33 g×3000 cm²/g/40 ml=100 cm²/ml).

Nanofibrous cellulose scaffold microcarriers were prepared by dilution of 0.5 g of dry microcarrier in 50 ml PBS. The fibers have a diameter of 500 nm and a length of 72 μm, and have a surface area of 50000 cm²/g (rounded from 53333 cm²/g for ease of approximations). The total surface area per volume was 500 cm²/ml (0.5 g×50000 cm²/g/50 ml=500 cm²/ml).

To present the same amount of surface area to the cells, 500 μl of Cytodex-1 solution was used (500 μl×100 cm²/ml=50 cm²) and 100 μl of nanofibrous cellulose scaffold solution was used (100 μl×500 cm²/ml=50 cm²).

To account for the unused space taken up by the inside of the microcarriers, the theoretical “dead volume” for each microcarrier was calculated (table 2).

Cytodex-1 beads have a radius of 95 μm; therefore, the surface of a single bead is:

$$A = 4 n \times {r}^{2} = 4 n \times {( 0.095 mm )}^{2} = 0.113 {mm}^{2} .$$

As the surface area of the beads is 3000 cm²/g, there are 2654867 beads/g (300000 mm²/g/0.113 mm²/bead). Cytodex-1 microcarriers were prepared at 3530973 beads in 40 ml, therefore 88274 beads/ml (resulting in a solution with 100 cm²/ml).

The volume of a Cytodex-1 bead (sphere) is:

$$V = 4 / 3 \times {r}^{3} = 4 / 3 \times n \times {( 0.095 mm )}^{3} = 0.0 \; 0359 {mm}^{3}$$

Therefore, the dead volume of 100 cm² of Cytodex-1 beads (88274 beads) is 317 μl/ml of media (88274 beads/ml×0.00359 μl/bead=317 μl/ml)

The diameter of the cellulose nanofibers is 500 nm and the length of the cellulose nanofibers is 72 μm. The surface area of a single fiber (cylinder) is:

$$A = 2 n \times {r}^{2} + 2 n \times r \times h = 2 n \times {( 0.00025 mm )}^{2} + 2 n \times ( 0.00025 mm ) \times ( 0.072 mm ) which is = 0.000113 {mm}^{2} .$$

As the surface area of fibers is 50,000 cm²/g, there are 4.4×10¹⁰ fibers/g (5000000 mm²/g/0.000113 mm²/fiber). Nanofibrous cellulose scaffold microcarriers were prepared at 2.2×10¹⁰ fibers in 50 ml, therefore 4.4×10⁸ fibers/ml (resulting in a solution with 500 cm²/ml).

The volume of a single fiber (cylinder) is:

$$V = n \times h \times {r}^{2} = n \times ( 0.072 mm ) \times {( 250 \times {10}^{- 6} mm )}^{2} = 1.4 \times {10}^{- 8} {mm}^{3} .$$

Therefore, the dead volume of 500 cm² of fibers (4.4×10⁸ fibers) is 6.16 μl/ml of media (4.4×10⁸ fibers/ml×1.4×10⁻⁸ μl/fiber). This gives a dead volume for 100 cm² of 1.23 μl/ml of media.

TABLE 2
Summary of physical properties of nanofibrous cellulose scaffolds and Cytodex-1.

| | Surface | | Dead volume | Dead volume |
|---|---|---|---|---|
| | area per | Surface area | per gram of | per 100 cm² of |
| | microcarrier | per gram | microcarrier | microcarrier |
| Microcarrier | (mm²) | (cm²/g) | (ml) | (μl) |
| Nanofibrous cellulose | 0.000113 | 53333 | 0.624 | 1.23 |
| scaffolds (Cellevate) | | | | |
| Cytodex-1 | 0.113 | 3000 | 9.5 | 317 |

### Seeding Cells on the Microcarriers

Cells were seeded on 50 cm² of each microcarrier (100 μl of the nanofibrous cellulose scaffold microcarrier solution or 500 μl of the Cytodex-1 solution) at a density of 10,000 cells/cm², 20,000 cells/cm², 40,000 cells/cm², 60,000 cells/cm² or 120,000 cells/cm² in 1 ml of cell culture media or 1.5 ml final volume for Cytodex-1 (to take account of dead volume of Cytodex-1).

Cells were incubated overnight (maximum 18 hours) at 37° C., 5% CO₂ in an incubator (no shaking). Falcon tube caps were not closed completely to enable gas exchange.

The next morning, 9 ml of cell culture media (DMEM, 10% FCS) was added to cells, pipetted up and down ten times with a 10 ml pipette to break the cell aggregates. The cells (in 10 ml media) were then transferred in a 10 cm non-coated Petri dish. The highest density was 600000 cells/ml in 10 ml media on 50 cm² (120,000 cells/cm²). The Petri dishes were then placed in the incubator at 37° C., 5% CO₂ on an orbital shaker at 60 rpm.

### Passaging Procedure and Cell Culturing

For long time culture (>3 days), cells were passaged. After 48 hours, cells were transferred in 125 ml Corning Flasks after pipetting up and down ten times with a 10 ml pipette to break the fiber aggregates. Fresh media (DMEM, 10% FCS) was added to the cells up to 30 ml. Nanofibrous cellulose scaffold or Cytodex-1 were added to the flasks (100 cm², which corresponds to 200 μl of nanofibrous cellulose scaffold microcarrier or 1 ml of Cytodex-1). For short time cell culturing and experiments (<3 days), cells were not passaged.

Cells were incubated at 37° C., 8% CO₂ on orbital shaker at 90 rpm. After 48 hours, additional 200 cm² of microcarriers were added with 20 ml of fresh media to reach 50 ml.

### Cell Viability

Cells were grown and passaged on nanofibrous cellulose scaffold microcarriers or Cytodex-1 as described above. At the indicated times, cells/fibers were pipetted ten times with a 10 ml pipette to break cells/fibers aggregate and obtain a homogenous suspension of cells/fibers in cell culture media. Then 500 μl of the suspension of cells/fibers media was mixed with 500 μl of 2× passive lysis buffer (Promega). The large volume aims to minimize the sampling effect and maximize the accuracy of the readings. The lysate was then centrifuged at 14000 rpm for 15 min at 4° C. Then 10 μl of the supernatant lysate was diluted in 90 μl of passive lysis buffer 1× (dilution 1 in 10. Then 5 μl of the diluted lysate was added to 25 μl of lysis buffer). The lactate dehydrogenase (LDH) activity was then analyzed according to the manufacturer's recommendation. The LDH activity is proportional to the number of cells as represented on the y-axis.

### Results

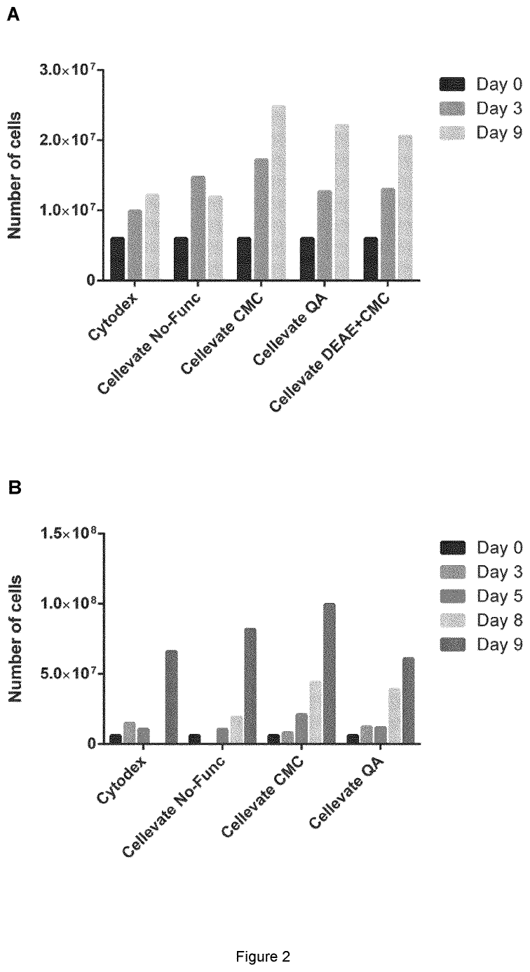

Cells attached better initially to the nanofibrous cellulose scaffolds than the market standard microcarrier (FIG. 1D-E). Both cell lines (HEK293AD and ARN8) proliferated fast and at high density on the nanofibrous cellulose scaffolds in the relevant time window of culturing, and in particular on the scaffolds with a functional moiety (FIG. 2A-B). All nanofibrous cellulose scaffolds performed better than the market standard benchmark.

It was possible to easily and cost effectively passage the cells on to the nanofibrous cellulose scaffolds. Especially, the fact that the passage of cells did not require trypsinization is beneficial as it reduces significantly cost, labour, and the risk of damaging or contaminating the cells.

Encouragingly, the nanofibrous cellulose scaffold allowed cells to grow for at least 48 hours at a density of 6×10⁶ cells per ml of medium (120000 cells/cm² on 50 cm² in 1 ml medium) compared to classic adherent culture which grow at 1×10⁶ cells per ml of medium.

### Conclusion

The nanofibrous cellulose scaffolds theoretically provides markedly more surface area per volume (approx. 250 times more) than the market standard microcarrier (Table 2). Therefore, in a finite volume (e.g. a bioreactor), the amount of available surface area that can be added is much higher for the nanofibrous cellulose scaffolds compared to the market standard Cytodex-1.

In this example this difference was “neutralised” and the same amount of surface area made available to the cells by adjusting the amount of microcarrier added. Even so, the cells grew best and at higher density on the nanofibrous cellulose scaffold, demonstrating that the native-like environment presented by the cellulose nanofibers are preferred by the cells.

### Example 3: Transient Cell Transfection and Protein Production on Nanofibrous Cellulose Scaffold

In this example transient transfection and subsequent protein production of cells grown on the nanofibrous cellulose scaffolds was assessed. Experiments were performed in two steps; a first where the surface area provided by the microcarrier was kept constant (as in example 2) and a second where the media volume was constant (thus allowing more nanofibrous cellulose scaffold in the culturing volume). The nanofibrous cellulose scaffolds were benchmarked against the market standard Cytodex-1.

### Method

The nanofibrous cellulose scaffolds were prepared as described in example 1. The following samples were assessed; Cellevate No-Func, Cellevate CMC, Cellevate QA, and Cellevate DEAE+CMC.

Transfection was performed using Polyethylenimine (PEI). Renilla Luciferase production was measured daily. The Luciferase activity is a marker of transfection and protein production. Pictures of cells cultured on nanofibers were provided using fluorescent microscopy to determine homogeneity of luciferase production. Quantity of Renilla luciferase protein was determined by Bright-Glo Luciferase assay (Promega).

Protein Production with Fixed Amount of Microcarrier Surface Area (50 mm²)

This experiment compared different types of microcarrier efficiency related to microcarrier surface area (50 mm²) by measuring protein production (luciferase) from HEK293AD and ARN8 cells.

Cells (HEK293AD and ARN8) were seeded at 6×10⁶ cells in 1 ml on 50 cm² of each microcarrier. Cells were transfected with 2.5 μg of Renilla luciferase reporter gene driven by the SV40 constitutive promoter using 7.5 μg of Polyethylenimine (PEI) as a transfection means.

Cells were grown and passaged on nanofibrous cellulose scaffolds or Cytodex-1 as described in example 2. At the indicated times, cells/fibers were pipetted ten times with a 10 ml pipette to break cells/fibers aggregate and obtain a homogenous suspension of cells/fibers in cell culture media. Then 500 μl of the suspension of cells/fibers media was mixed with 500 μl of 2× passive lysis buffer (Promega). The large volume aims to minimize the sampling effect and maximize the accuracy of the reading.

Luciferase activity was analyzed 24 hours and 48 hours after transfection using the Bright-Glo Luciferase assay (Promega). Cells and microcarrier were homogenized by pipetting and 50 μl of media was taken to mix with 50 μl Bright-Glo reagent, luciferase activity was immediately analyzed on a luminometer (Promega) according to the manufacturer's procedure.

Protein Production with Fixed Volume of Microcarrier

This experiment compared different types of microcarrier efficiency related to microcarrier volume by measuring protein production (luciferase) from HEK293AD cells in a finite volume of cell culture media.

Microcarriers were seeded at the same cell density (20,000 cells per cm²) to compare luciferase production after transient transfection of a same volume of microcarrier (100 mm³) in 2 ml of cell media.

Cells were transfected with 40,000 molecules of luciferase reporter gene driven by SV40 promoter using 7.5 μg of Polyethylenimine (PEI) as a transfection means. Five hours after transfection, 3 ml of fresh media was added to the 50 ml Falcon tube (cap is not closed tight to allow O₂/CO₂ exchange). Luciferase activity was analyzed 24 hours and 48 hours after transfection using Bright-Glo Luciferase assay (Promega). Cell microcarrier was homogenized by pipetting and 50 μl of media was taken to mix with 50 μl Bright-Glo reagent, Luciferase activity was immediately analyzed on the luminometer (Promega).

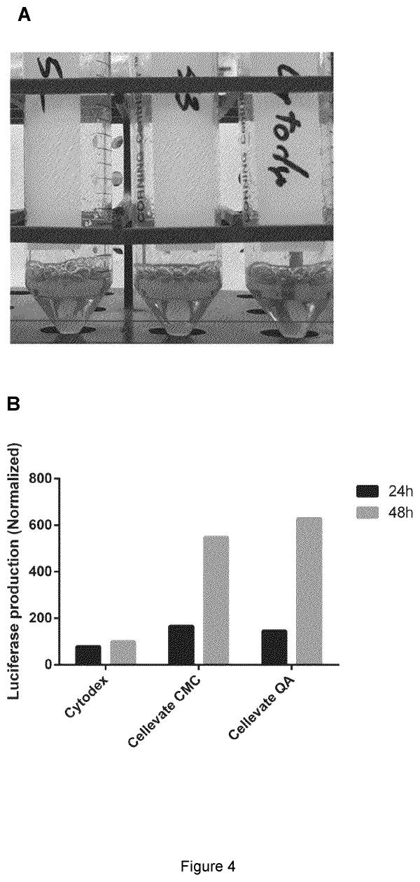

Importantly, it was noticed that the volume of the Cellevate microcarrier is significantly bigger when wet. The volume per cm² of the Cellevate microcarrier fibers is, theoretically, about 250 times smaller than the volume per cm² of the Cytodex-1 microcarrier as calculated in example 2. To obtain an actual value, a series of experiments was conducted to estimate the volume of the hydrated nanofibrous cellulose scaffolds. It was found that estimating that the volume occupied by the nanofibrous cellulose scaffold is 5 times smaller than that of Cytodex-1 is a good approximation (FIG. 4A).

The wet volume for Cytodex-1 is 317 mm³ for a surface area of 100 cm² (32 cm²/100 mm³). If the volume of wet nanofibrous cellulose scaffold microcarrier would be 5 times smaller than Cytodex-1, then the volume of the wet nanofibrous cellulose scaffold microcarrier would be 100 mm³ for 160 cm². Each microcarrier was seeded at the same cell density: 20,000 cells per cm² in a volume of 100 μl of carrier in a final volume of 5 ml in a 50 ml falcon tube (cap was not closed tight to allow O₂/CO₂ exchange).

### Results

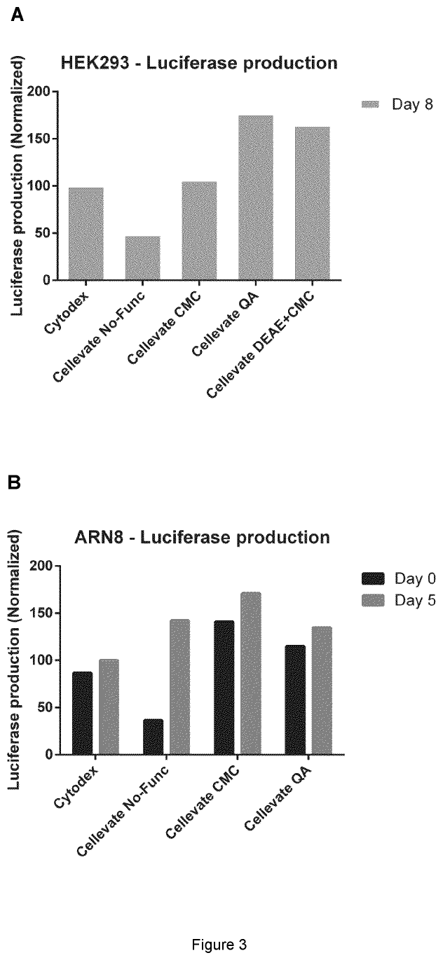

When using the same amount of available surface area, HEK293AD cells produced approximately 70% more luciferase per ml on Cellevate CMC and Cellevate QA than on Cytodex-1 8 days after transfection (FIG. 3A). ARN8 cells produced 40-70% more luciferase per ml on nanofibrous cellulose scaffold microcarriers (Cellevate No-Func, Cellevate CMC and Cellevate QA) compared to Cytodex-1 5 days after transfection (FIG. 3B).

By decanting of the microcarriers, it was observed that a good approximation to reach equal volumes of microcarriers was that the volume of the nanofibrous cellulose scaffold microcarrier was 5 times smaller than the volume of the Cytodex-1 (FIG. 4A). Thus, the volume of added nanofibrous cellulose scaffold microcarrier was adjusted accordingly.

Assessment of cell growth when adding a fixed volume of microcarrier demonstrated that the significantly reduced dead volume of the nanofibrous cellulose scaffold and thereby the increased available surface area in a finite cell culturing vessel is highly advantageous. The yield of luciferase from HEK293AD cells on the nanofibrous cellulose scaffold microcarriers (Cellevate CMC and Cellevate QA) was approximately 5.5-6.3 times higher after 48 hours as compared to Cytodex-1 (FIG. 4B).

### Conclusion

This example demonstrates that the nanofibrous cellulose scaffold microcarriers enable transient transfection of HEK293AD and ARN8 cells with high efficiency leading to high yield production of active recombinant protein. Moreover, the high surface area per volume of the nanofibrous cellulose scaffold microcarriers allows an increased amount available surface area to be packed in a finite volume, such as a bioreactor. This example show that this advantage leads to significantly improved yield of recombinant protein.

### Example 4: Processing of the Initial Cellulose Nanofiber Material

In this example various methods of dividing the initial cellulose nanofiber material were tested and their influence on the nanofibrous cellulose scaffold was assessed.

### Method

Preparation of the initial cellulose nanofiber material was performed as described in example 1.

### Processing Protocols

Different methods of dividing the initial cellulose nanofiber material were evaluated. Thus, samples were prepared according to the following:

### Mechanical Cutting (by Scissor):

2.5 g Cellulose sheets were cut up in 10×10 mm pieces with a pair of scissors and assayed directly as fragmented cellulose sheets.

### Blending:

2.5 g cellulose sheets were cut up in 20×20 mm pieces with a pair of scissors and added together with 250 ml water to a lab mixer (LB20, Waring Laboratory). The cellulose nanofiber material was processed at 7000 rpm for varying durations of time (0.5, 1, 2.5, 5, and 10 min).

### Laser Cutting:

Cellulose sheets were cut with a laser cutter (Epilog laser, Zing 24) in three different sizes (0.75×0.75 mm, 1.5×1.5 mm and 3×3 mm). The cut sheets were assayed as is.

### Dispersing:

2.5 g cellulose sheets were cut up in 20×20 mm pieces with a pair of scissors and added together with 250 ml water to an Erlenmeyer flask. The sample was processed using a disperser (IKA T25 digital Ultra-Turrax with S25 NB—25 G disperser tool) at 18000 rpm for varying durations of time (0.5, 1, 2.5, 5, 10, 15 and 60 min).

### Microscopy

Samples of processed cellulose nanofiber material were diluted 10× in water and added to a glass slide. Images of the network of cellulose nanofibers in the sample were captured on a light microscope (Leica) using 40× magnification.

### Scanning Electron Microscopy (SEM)

Samples of processed cellulose nanofiber material were diluted 1000-10000× in water and a droplet of sample is applied to a SEM fixture. The samples were dried and sputter coated with gold before capturing images on a Hitachi SU3500. Images were captured at different magnifications.

### Fiber Length Measurements

Fiber length of the cellulose nanofibers were determined either by SEM (Hitachi SU3500) or by light scattering (Malvern Mastersizer S).

SEM images were evaluated manually by visual inspection to ensure that all measured fibers had both endings visible. The fiber lengths were determined by use of ImageJ software. Fiber lengths from several SEM images were determined to get a larger dataset.

Fiber lengths were also determined using light scattering. Briefly, 1 ml of sample was added in water to the Malvern Mastersizer S and measurements were performed with the settings described under the definition of “mean length”. Sample was added to the sample container until the obscuration value was between 15-20%. From each sample a fiber length histogram displaying the fiber length distribution was generated. The statistics of the distribution are calculated from the results using the derived diameters D[m,n]—an internationally agreed method of defining the mean and other moments of particle size. D(v, 0.5), D(v, 0.1) and D(v, 0.9) are standard “percentile” readings from the analysis. D(v, 0.5) is the fiber length at which 50% of the sample is smaller and 50% is larger than this length. This value is also known as the Mass median diameter (MMD) when used for particles. D(v, 0.1) is the fiber length for which 10% of the sample is below this length. D(v, 0.9) gives a fiber length for which 90% of the sample is below this length. The volume weighted mean fiber length D[4,3] was also determined.

### Pipetting/Floating Test

Samples of processed cellulose nanofiber material were tested for their ability to be pipetted. 1 ml of sample was pipetted out of the sample container and subsequently expelled from the pipette tip into a tube with water. The ease of pipetting was evaluated, including the propensity for processed cellulose nanofiber material to clog the pipette.

Processed cellulose nanofiber material transferred to a tube containing water was then assessed for its propensity to float. Samples were vigorously shaken and it was observed by visual inspection whether the material floated immediately after and 24 hours after shaking.

### Results

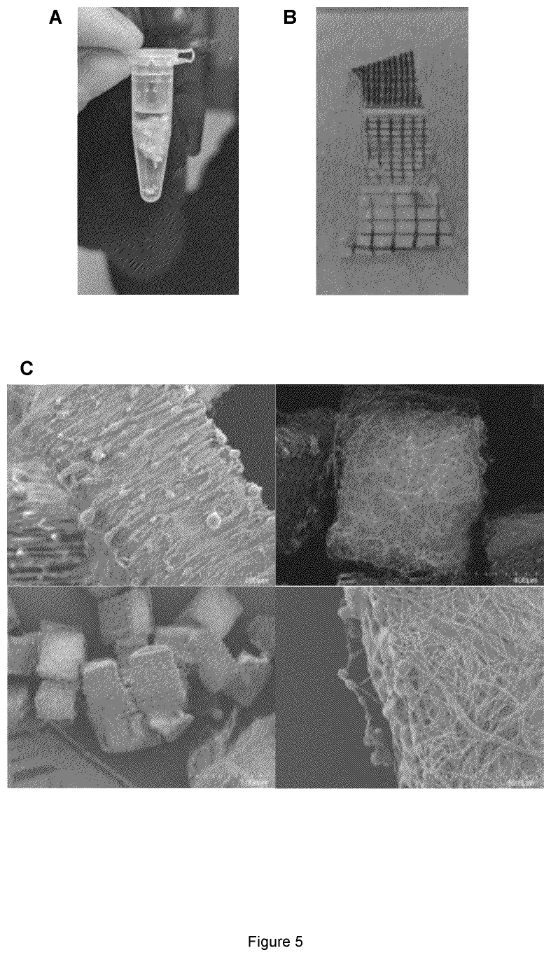

The pipetting test of scissor cut and laser cut cellulose sheets showed that these cut pieces are too large to pipette as they will clog the pipette tip. Also, cellulose nanofiber material processed in this manner generates pieces of dimensions that trap air bubbles and float in solution (FIG. 5A). Furthermore, cutting with a laser makes the cellulose nanofibers melt, burn and stick together and is therefore not a suitable operation for dividing the cellulose nanofibers (FIG. 5B-C).

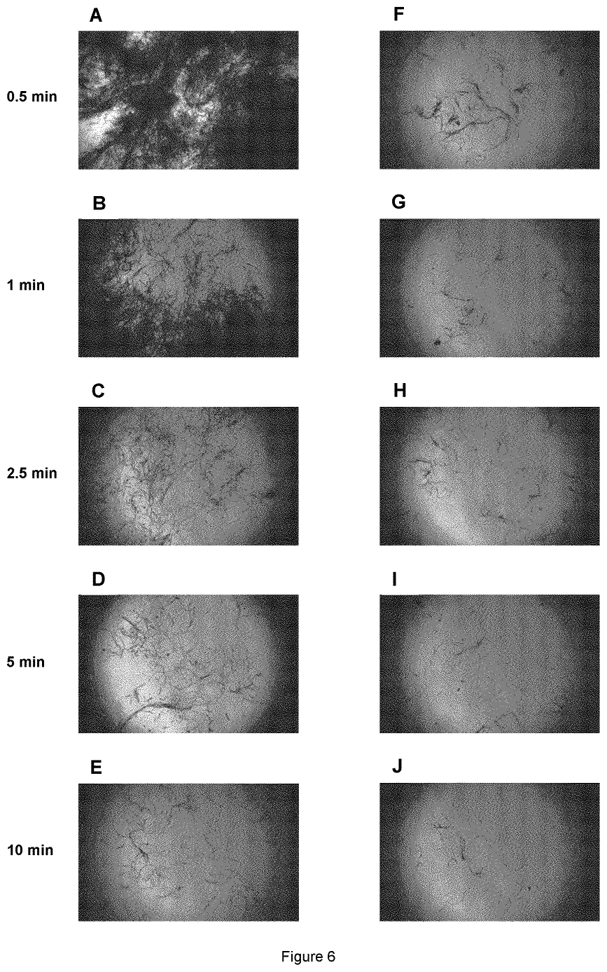

Cellulose nanofiber material processed by the blender (FIG. 6A-E) generally produced more dense and entangled samples with less homogenously distributed cellulose nanofibers than cellulose nanofiber material processed by the disperser (FIG. 6F-J). It appeared that cellulose nanofiber material dispersed for at least 2.5 min was more homogenously distributed throughout the sample and with fewer long cellulose nanofiber strands (FIG. 6F-J). Thus, a minimum of e.g. 2 min of dispersing may be advantageous to avoid excess entanglement and clusters of cellulose nanofibers.

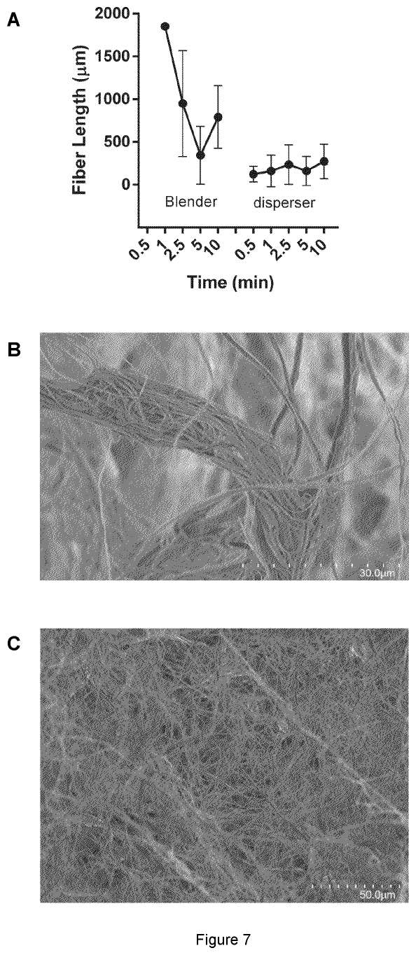

SEM images of the processed cellulose nanofiber material confirmed that the samples processed by blending produced longer (FIG. 7A) and more entangled cellulose nanofibers (FIG. 7B-C) than processed cellulose nanofiber material processed by dispersing.

The dispersing time influenced the mean length of the cellulose nanofibers in the cellulose nanofiber material. Shorter fiber lengths were obtained for longer dispersing times. Shorter fibers may be advantageous as they are less prone to form cluster and entangle, thereby reducing the amount of non-accessible surface area and the risk of clogging. A summary of the determined mean fiber lengths is given in table 3.

TABLE 3
Fiber length measurements from Malvern Mastersizer S.

| | D[v, 0.1] (μm) | D[v, 0.5] (μm) | D[v, 0.9] (μm) | D[4, 3] (μm) |
|---|---|---|---|---|
| Dispersing | 10% below | 50% below | 90% below | Volume |
| time (min) | this value | this value | this value | weighted mean |
| 0.5 | 12 | 195 | 549 | 242 |
| 1 | 9 | 137 | 482 | 198 |
| 2.5 | 6 | 84 | 343 | 135 |
| 5 | 5 | 71 | 306 | 118 |
| 10 | 5 | 64 | 290 | 110 |
| 15 | 5 | 56 | 256 | 97 |
| 60 | 4 | 42 | 192 | 72 |

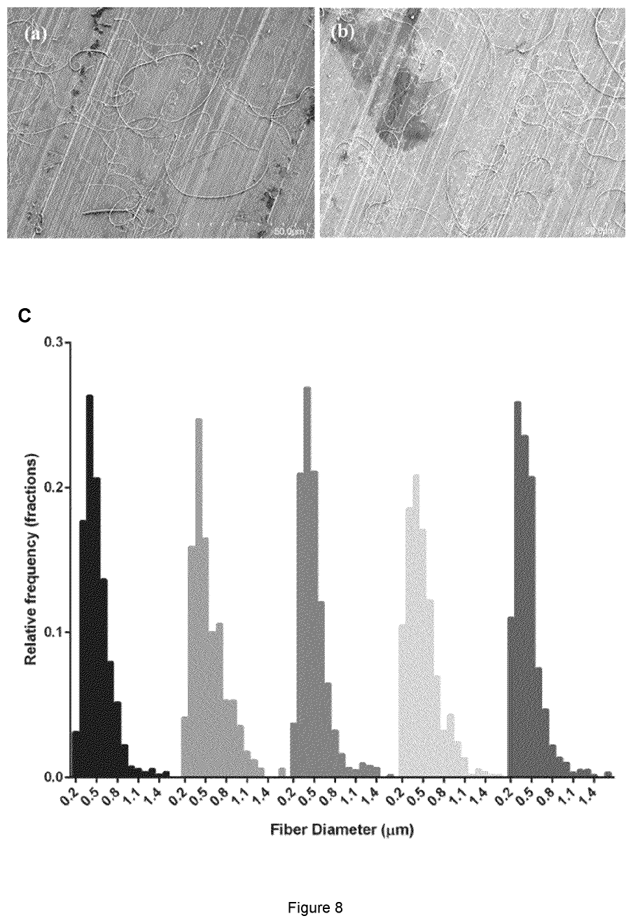

SEM measurements were used to determine cellulose nanofiber diameter (FIG. 8A-B). More than 2000 individual cellulose nanofibers were measured using ImageJ software and gave a mean cellulose nanofiber diameter of 500 nm (FIG. 8C).

None of the cellulose nanofiber materials processed by dispersing trapped air bubbles or had any tendency to float in solution. Pipetting of these samples was least challenging for samples that had been dispersed for longer durations of time. As such it was preferred to disperse for at least 10 min to improve flow through the pipette.

### Conclusion

This example demonstrates that not every method for dividing the initial cellulose nanofiber material is equally effective and suitable for preparation of the nanofibrous cellulose scaffold. In particular, dispersing is advantageous as it homogenously divides the cellulose nanofiber material without generation of entanglements or clusters.

### Example 5: Processing Nanofibers of Material Different from Cellulose

In this example the processing of a variety of nanofibers different from cellulose were assessed with the aim of identifying auxiliary nanofibers suitable for preparation of nanofibrous scaffolds for growing cell cultures on. The electrospun nanofibers were characterized by scanning electron microscopy (SEM) and by visual inspection (following dividing of the nanofibers).

### Method

### Electrospinning

Nanofibers of polycaprolactone (PCL), poly-L-lactic acid (PLA), a mixture of PCL and PLA (PLA/PCL) and cellulose were electrospun. Electrospinning was performed on a Fluidnatek LE50 apparatus as described in Example 1.

PCL fibers were obtained by dissolving 8% Polycaprolactone pellets (Sigma Aldrich, MW 80000), in Chloroform:Methanol, 1:1 solution. Needle to collector distance was set to 20 cm, flow rate to 3 ml/h, and voltage to 18 kV.

PLA fibers were obtained by dissolving 8% PLLA pellets (Goodfellow, MFR:24), in Chloroform:Methanol, 3:2. Needle to collector distance was set to 24 cm, flow rate to 4.5 ml/h and voltage to 35 kV.

PLA/PCL fibers were obtained by dissolving PLA pellets (Goodfellow, MFR:65) and PCL pellets (Sigma Aldrich, MW 80000) 1:2 to an 8% polymer solution in Chloroform: Methanol, 3:2. Needle to collector distance was set to 20 cm, flow rate to 3 ml/h and voltage to 18 kV 1 ml of polymer solution was spun for each sheet of fibers.

### Scanning Electron Microscopy

The electrospun material of PCL, PLA, mixed PCL/PLA and cellulose were imaged on a Hitachi SU3500 as described in Example 1. Images of the nanofiber materials were obtained for both undivided and divided nanofibers.

### Processing of PCL, PLA and PCL/PLA Nanofiber Material

Electrospun sheets of PCL, PLA and mixed PLA/PCL were cut into smaller 10×10 mm pieces and mixed with two different mixing tools for 5 minutes. The cut sheets were mixed either with a blender at 7000 rpm (LB20E Laboratory blender, Waring) or with a disperser at 18000 rpm (IKA T25 digital Ultra Turrax).

### Results

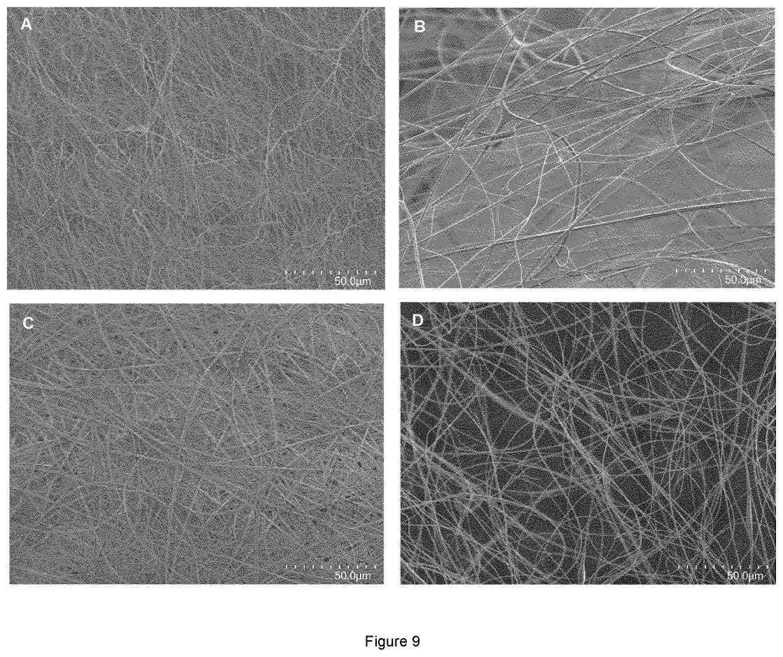

Nanofibers could be electrospun from all materials (PCL, PLA, PLA/PCL, and cellulose) and formed nanofibrous sheets (see FIG. 9A-D).

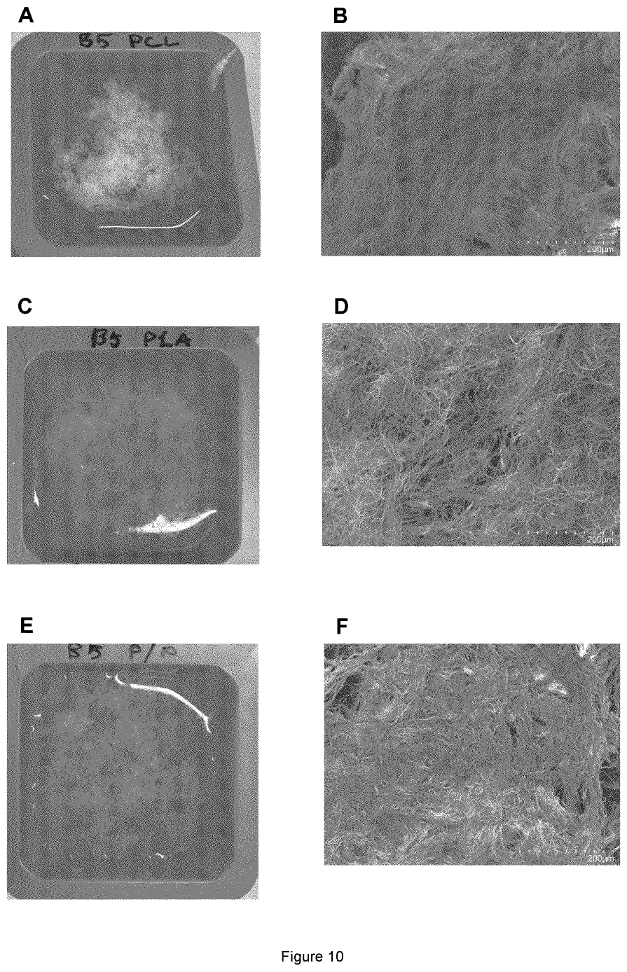

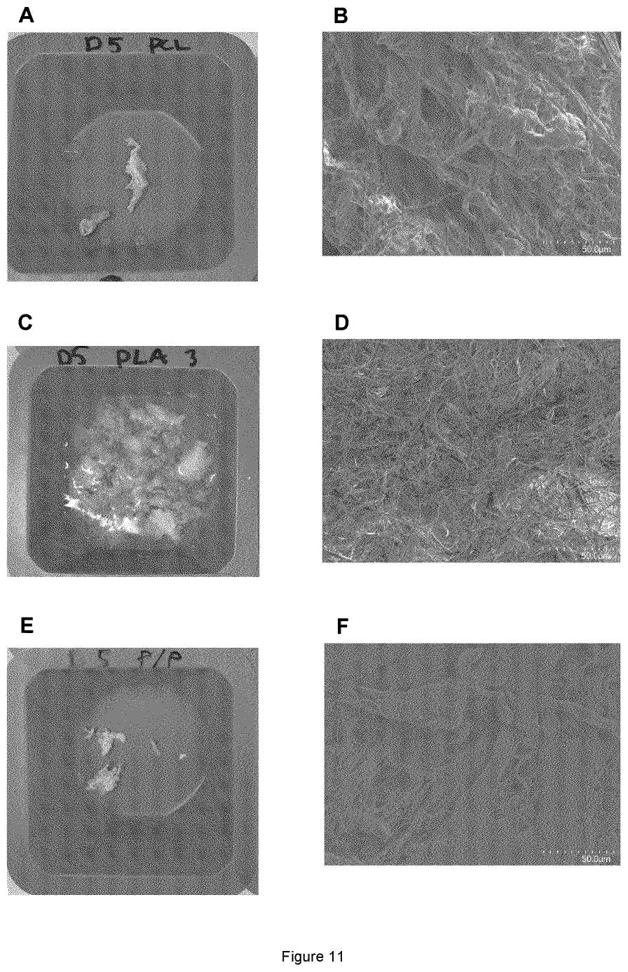

The sheets made from nanofibers different from cellulose were subjected to two individual modes of dividing the nanofibers, namely blending (FIG. 10A-F) and dispersing (FIG. 11A-F). The tests showed that it was not possible to uniformly blend or disperse either PCL, PLA, or PLA/PCL nanofibers into a homogenous mixture of short strand nanofibers.

Both the PCL nanofibers (FIG. 10A-B and FIG. 11A-B) and the PLA/PCL nanofibers (FIG. 10E-F and FIG. 11E-F) melted or deformed during the process. In particular, the nanofibers melted together to form either large pieces of solid polymer or large entangled clusters of nanofibers with semi-melted nanofibers.

The PLA nanofibers (FIG. 10C-D and FIG. 11C-D) did not melt in the same way as the PCL fibers, but all materials did easily get stuck in the mixing tool or got entangled on the blades of the blender and halted the processing.

### Conclusion

This example demonstrates that it is not possible to uniformly blend or disperse nanofibers of all materials. Accordingly, not all nanofiber materials can be easily transformed into shorter strands followed by formation of a nanofibrous scaffold as described herein. Thus, it is preferred to use cellulose nanofibers for the preparation of the nanofibrous scaffolds.

### Example 6: Means of Dividing Initial Cellulose Nanofibers

In this example the influence of the mode of dividing the nanofibers on the length of the cellulose nanofibers was assessed. The nanofibers were divided by either blending or dispersing and the length of representative fractions of nanofibers were measured by individual analysis of SEM images or in solution by laser diffraction.

### Method

QA-functionalized cellulose nanofibers were prepared for further testing. Cellulose acetate sheets were prepared by electrospinning as described in Example 1.

### Regeneration of Cellulose

Cellulose was obtained by regeneration (deacetylation) of electrospun cellulose acetate sheets. Briefly, 25 g cellulose acetate sheets were cut into smaller pieces (2×2 cm) and added to a beaker with 1.25 L of 0.5 M NaOH in 95% ethanol solution. The cellulose fibers were regenerated 24 hours at room temperature. After completion of reaction time, the regenerated cellulose nanofibers were filtered through a Buchner filter, immersed in 500 mL dH2O for 1 minute and filtered again. This process was repeated until the conductivity of the final washing step was 0 μS/cm to make sure there were no residual NaOH, or acetate left. Finally, the nanofibers were dried in the oven over night at 60° C.

### Dividing of Cellulose Nanofibers

A 1% cellulose suspension was prepared by adding 5 g of regenerated cellulose to 500 ml of distilled water. The solution was added to the disperser and mixed for various timepoints at 18,000 rpm (IKA T25 digital Ultra Turrax). 100 mL samples labeled were removed after 1 (D1), 5 (D5), 15 (D15) and 60 (D60) minutes of mixing. The procedure was replicated with blender, but with the speed set to 7,000 rpm (LB20E Laboratory blender, Waring) (samples labeled: B1, B5, B15 and B60). After mixing, the fibers were dried in the oven over night at 60° C. The size distribution of the nanofibers was analysed by SEM and dynamic light scattering (DLS). SEM measurements and DLS measurements were performed as described in Example 4. The length of individual cellulose nanofibers was assessed from SEM images (4 induvial 10 μL drops for each timepoint). For the light scattering experiments, 10.000 fibers were measured per sample.

### Functionalization of Cellulose Nanofibers

Samples of regenerated cellulose fibers, blended or dispersed at various timepoints were functionalized with quaternary ammonia (QA). Briefly, 1 g of regenerated cellulose from each time point was resuspended in 50 ml of 1.5 M NaOH solution and mercerised for 2 hours under stirring at room temperature. After the mercerization step, the temperature was increased to 80° C. and 2.5 ml CHPTAC was added dropwise. The reaction continued at 80° C. for 4 hours. After the reaction, the mixture was filtered through a Buchner filter, immersed in 200 mL dH₂O for 1 minute and filtered again. This process was repeated until the conductivity of the final washing step was 0 μS/cm to make sure there were no residual CHPTAC or NaOH left. Finally, the filtered fibers were resuspended in water to a 2% solution and freeze dried for further experimentation.

### Results

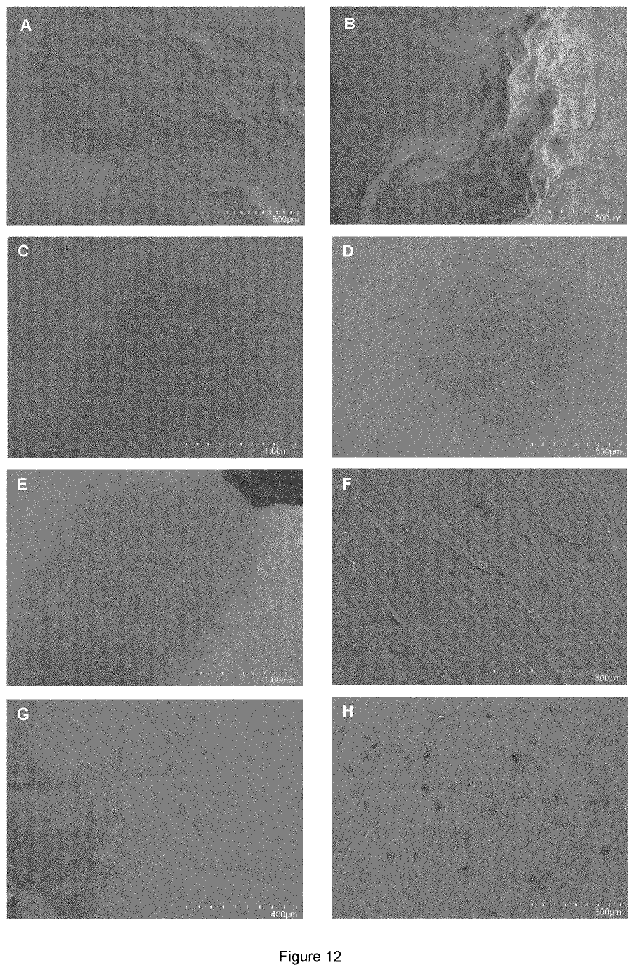

It is clear from the SEM images (FIG. 12A-H) that the disperser quickly provides a homogenous nanofiber population with relatively few long nanofibers and without any significant entanglement. While large chunks of uncut nanofibers are present in the samples after only 1 min of dividing (FIG. 12A-B), the disperser presents a finer population of nanofibers with only few smaller chunks of entangled nanofibers already after 5 min of dispersing (FIG. 12D). After 15 and 60 min of dispersing the presence of entangled nanofibers is almost completely eliminated (FIGS. 12F and 12H). In contrast, the blended samples comprise large chunks of entangled fibers even after 60 min of blending (FIG. 12G).

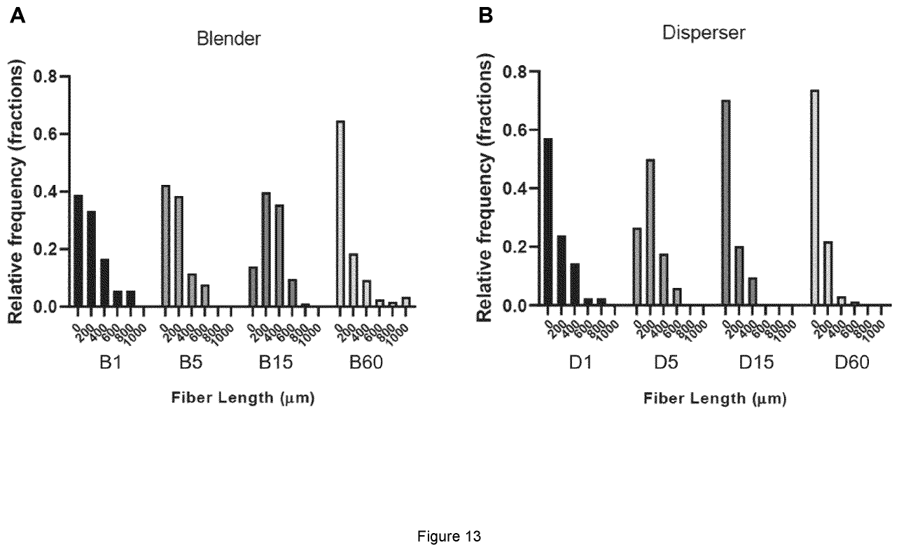

The data are summarised for each of the samples in FIG. 13A-B. It is clear that the frequency of long nanofibers is higher in the blended samples compared to the dispersed samples. Furthermore, there is a tendency towards longer processing times producing less long nanofibers.

This relative trend is supported by measurements of the nanofibers using light diffraction. These data are summarised in Table 4.

TABLE 4
Fiber length measurements of cellulose nanofibers prepared by
blending (B1, B5, B15, B60) and dispersing (D1, D5, D15, D60).
Measurements were performed on a Malvern Mastersizer S.

| | D[v, 0.1] (μm) | D[v, 0.5] (μm) | D[v, 0.9] (μm) | D[4, 3] (μm) |
|---|---|---|---|---|
| | 10% below | 50% below | 90% below | Volume weighted |
| Sample | this value | this value | this value | mean |
| B1 | 34 | 305 | 665 | 328 |
| B5 | 7 | 116 | 465 | 185 |
| B15 | 3 | 55 | 257 | 96 |
| B60 | 3 | 43 | 252 | 87 |
| D1 | 23 | 236 | 612 | 280 |
| D5 | 4 | 60 | 259 | 98 |
| D15 | 3 | 49 | 237 | 87 |
| D60 | 3 | 46 | 230 | 83 |

Overall it is desired that the mean length of the cellulose nanofibers is reduced. However it is also important that the population of nanofibers does not contain many long nanofibers as these may act as nucleus for larger clusters of nanofibers and entanglements. These clusters or entanglements of nanofibers are undesirable in the nanofibrous scaffold as large fractions of the surface area remains inaccessible to the cells seeded thereupon. Moreover, an inhomogeneous population of nanofibers comprising clusters and entanglements are undesirable because the variability between batches of the nanofibrous scaffold becomes inconsistent and therefore unreliable for cell growth.

### Conclusion

This example demonstrates that the means of dividing the cellulose nanofiber material influences the size distribution of the nanofibers, and in particular the fraction of longer fibers. Dispersing of the nanofibers is preferred because it rapidly removes longer fibers and yields the most homogenous population of cellulose nanofibers.

### Example 7: Degree of Functionalization and Cell Growth on Cellulose Nanofibers

In this example the influence of functionalization and dispersing time of the nanofibers on the cell growth on the resulting nanofibrous scaffold was investigated.

### Method

### Sample Preparation

Samples were prepared by weighing up 0.05 g freeze dried cellulose samples (prepared as described in Example 6) in 50 ml tubes. To each tube 25 ml distilled water was added and the fibers were allowed to swell for 2 hours. After the swelling, the tubes were centrifuged, and the supernatant discarded.

For evaluation of influence of degree of functionalization on cell growth, samples were prepared by addition of various amounts of CHPTAC, thereby yielding different ion exchange capacities.

For saturation of the exchange sites with chloride ions, the samples were incubated under agitation with 25 ml 0.1 M HCl for 1 hour. After the incubation, the samples were filtered using a 0.2 μm Millipore filter unit and the supernatant discarded.

To remove unbound chloride ions, the nanofibrous cellulose microcarriers were incubated with 25 ml 0.1 mM HCl for 10 min. The microcarriers were then centrifuged and the supernatant discarded. To displace the bound chloride ions with sulfate ions, the microcarriers were incubated for 2 hours with 40 ml of 10% (w/w) sodium sulfate solution.

The microcarriers were subsequently filtered using a 0.2 μm Millipore filter unit and the filtrate was saved for silver nitrate titration.

### Ion Exchange Capacity Measurement

The ionic capacity for each sample was determined by AgNO₃ titration using a Mettler Toledo T5 Titrator. 10 ml of each filtrate collected from the last sample preparation step above, was diluted in 30 mL distilled water. The samples were then titrated with 0.01 M AgNO₃ to determine the amount of chloride ions that were bound to the functionalized cellulose fibers. From the equivalence point on each titration curve, the ion exchange capacity (mmol Cl⁻/g) was calculated using the LabX software.

### Cell Culture Protocol

Cell growth experiment on cellulose fibers was performed in 12 well plate format using HEK293 cells (ATCC CTRL-3216) at passage 8 (92% viability on the day of inoculum with fibers).

1 mg of cellulose fibers for each condition were washed 3 times with DPBS and resuspended in 10 ml of cell culture media (DMEM with high glucose and GlutaMAX Supplement and pyruvate, 10% heat-inactivated FBS and 1% penicillin-streptomycin). 300000 cells per well were seeded in 0.3 ml of media with 0.3 ml (corresponding to 0.3 mg) of QA-functionalized cellulose fibers prepared as described in Example 6 (D1, D5, D15 and D60) and left on an orbital shaker (16 mm) at 35 rpm overnight in an incubator (at 37° C. with 5% CO₂).

The morning after, each well was topped off with 1.4 ml media to reach a final volume of 2 ml and bringing the initial cell concentration before analysis up to 150000 cells/ml. The shaker rotation speed was increased to 65 rpm.

24, 48 and 72 hours after seeding, three replicates from separate wells from each timepoint were taken for further analysis. Enzymatic dissociation of cells was used in combination with the microcarrier program of the NucleoCounter (NC-202) to assess the cell density and viability over time.

### Results

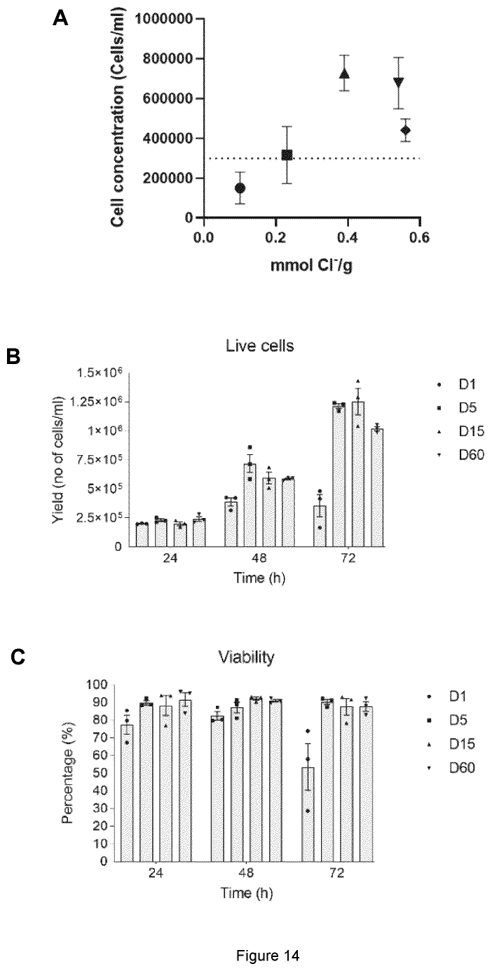

Cell growth was enhanced on nanofibrous cellulose scaffolds with higher degree of functionalization (FIG. 14A). In particular, it appeared that best cell growth was achieved for cellulose microcarriers with an ion exchange capacity above 0.3 mmol Cl⁻/g.

Consistent cell growth was achieved on all cellulose microcarriers. The data show that cells grown on cellulose microcarriers prepared from cellulose nanofibers that had been dispersed for at least 5 min proliferated best and showed great viability (FIG. 14B-C). Cell densities reached an eight-fold increase over the initial inoculum density over 72 hours of incubation. This was achieved without transferring of cells or change of media.

### CONCLUSION

This example demonstrates that the cellulose microcarriers could be efficiently functionalised and that a minimum of ion exchange capacity is preferred to enhance cell growth. Cellulose microcarriers wherein the nanofibers had been dispersed for more than 1 min (such as 5 min) constituted the best scaffolds for cell growth. Without being bound by theory, it is contemplated that the reduction of nanofiber clusters and entanglement ensures that a larger surface area is made available for the cells to be seeded on and interact with.

1. A method of preparing a nanofibrous cellulose scaffold, said method comprising:
dividing an initial cellulose nanofiber material into a processed cellulose nanofiber material,
functionalizing the processed cellulose nanofiber material by addition of a reagent comprising a functional moiety, and
drying the processed cellulose nanofiber material,
wherein the dividing is achieved by dispersing,
thereby providing the nanofibrous cellulose scaffold.

2-15. (canceled)

16. The method according to claim 1, wherein the dispersing is performed with a high-speed disperser.

17. The method according to claim 1, wherein the dispersing is performed for at least 2 min.

18. The method according to claim 1, wherein the mean length of the cellulose nanofibers in the processed cellulose nanofiber material is in the range of about 70 μm to about 120 μm.

19. The method according to claim 1, wherein the initial cellulose nanofiber material is prepared by electrospinning, meltblowing, drawing, self-assembly, template synthesis, and thermal-induced phase separation.

20. The method according to claim 1, wherein the drying comprises freezing or lyophilization of the processed cellulose nanofiber material.

21. The method according to claim 1, wherein the functional moiety is a chemical moiety selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

22. The method according to claim 21, wherein the functional moiety is quaternary ammonium (QA).

23. The method according to claim 1, wherein the ion exchange capacity of the nanofibrous cellulose scaffold is at least about 0.3 mmol Cl⁻/g.

24. A nanofibrous cellulose scaffold obtainable from a method according to claim 1.

25. A nanofibrous cellulose scaffold comprising a cellulose nanofiber material with a mean length of the cellulose nanofibers of less than about 250 μm, and wherein the cellulose nanofiber material is functionalized with a functional moiety.

26. The nanofibrous cellulose scaffold according to claim 25, wherein the nanofibrous cellulose scaffold has a BET surface area of at least about 40000 cm²/g.

27. A microcarrier comprising a nanofibrous cellulose scaffold according to claim 25.

28. A cell culturing device comprising a container loaded with a nanofibrous cellulose scaffold according to claim 25.

29. A method for cell culturing comprising:
(i) providing the cell culturing device according to claim 28,
(ii) adding a composition comprising a cell population to the cell culturing device,
(iii) incubating the cell population to provide a proliferated cell population, and
(iv) optionally, extracting the proliferated cell population from the cell culturing device.
